(19) United States
(12) Patent Application Publication
(10) Pub. No.: US 2019/0185877 A1
(43) Pub. Date: Jun. 20, 2019

(54) ANTI-MICROBIAL PROTEINS

(71) Applicant: Monsanto Technology LLC, St. Louis, MO (US)

(72) Inventors: Patrick Boyle, St. Louis, MO (US); David Caldwell, St. Louis, MO (US); Jaishree Chittoor, Wildwood, MO (US); Jintai Huang, Chesterfield, MO (US); Susanne Kjemtrup, St. Louis, MO (US); Gerrit Segers, Wildwood, MO (US)

(21) Appl. No.: 16/070,733

(22) PCT Filed: Jan. 19, 2017
(86) PCT No.: PCT/US2017/014153

§ 371 Date: Jul. 17, 2018

Related U.S. Application Data
(60) Provisional application No. 62/280,597, filed on Jan. 19, 2016.

(51) Int. Cl. C12N 15/82; C07K 14/415

(52) U.S. Cl. CPC C12N 15/8282; C07K 14/415

(57) ABSTRACT

The disclosure provides polynucleotide molecules encoding novel defensins conferring increased pest tolerance and/or pesticidal activity when expressed in a plant, and recombinant DNA constructs and vectors comprising these molecules. Methods of making transgenic plants comprising recombinant defensin-encoding polynucleotide molecules and constructs, and transgenic plants, plant parts and seeds produced by these methods are provided. Compositions comprising one or more novel defensins of the disclosure are also provided having pesticidal and/or anti-microbial activity, as well as methods of their use.

### REFERENCE TO RELATED APPLICATION

This application claims the benefit of U.S. Provisional Application No. 62/280,597, filed Jan. 19, 2016, which is herein incorporated by reference in its entirety.

### FIELD OF THE INVENTION

The invention relates to the field of agricultural biotechnology. More specifically, the invention relates to nucleotide and polypeptide molecules, DNA constructs, and methods for producing plants with improved microbial pathogen or pest tolerance, as well as transgenic plants with improved pesticidal and/or fungicidal activity. The invention further relates to pesticidal and/or fungicidal compositions.

### INCORPORATION OF SEQUENCE LISTING

A sequence listing contained in the file named “MONS388WO_ST25.txt” which is 592 KB (measured in MS-Windows®) and created on Jan. 18, 2017, is filed electronically herewith and incorporated by reference in its entirety.

### BACKGROUND

Control of fungal pathogens and other disease-causing microbes that affect plants is one of the major difficulties facing the agricultural and horticultural industries. Although chemical agents have been successfully employed, a range of environmental and regulatory concerns is associated with the continued use of chemical approaches to control plant pests. Furthermore, with increased use of chemical pesticides comes increased resistance to these chemicals in pathogen and pest populations. Thus, further investigation to discover alternative mechanisms of providing resistance in plants to pathogens, such as insects, nematodes, microorganisms, fungi, bacteria and viruses, is needed.

### SUMMARY

In one aspect, the invention provides a recombinant DNA construct comprising a nucleic acid sequence encoding a multi-domain defensin polypeptide comprising a first defensin region connected to a second defensin region by a linker region, the first defensin region and the second defensin region each comprising a gamma-thionin domain, wherein the nucleic acid sequence encoding the defensin polypeptide is operably linked to a promoter functional in a plant cell.

In another aspect, the invention provides a recombinant DNA construct comprising a nucleic acid sequence encoding a single domain defensin polypeptide.

For the multi-domain defensin, in some embodiments, the first defensin region is heterologous with respect to the second defensin region or the linker region, while in other embodiments the first defensin region or the second defensin region is heterologous with respect to the linker region. In certain embodiments, the first defensin region is identical to the second defensin region. In other embodiments, the first defensin region is different from the second defensin region. In certain embodiments, the promoter may be a heterologous promoter. In further embodiments, each defensin domain comprises at least 6 cysteine residues. The multi-domain defensin may be at least 90 amino acids in length. Each defensin domain may comprise: (a) between 5 and 10 cysteine residues; (b) a Pfam gamma-thionin domain (PF00304) with an E-value cutoff of 1e⁻³; or (c) a Cys-stabilized αβ (CSαβ) motif.

In certain embodiments, the first defensin region or the second defensin region of a multi-domain defensin comprises a polypeptide having at least 80% identity, at least 90% identity, at least 95% identity, or at least 97% identity to an amino acid sequence selected from the group consisting of: SEQ ID NOs: 559-662, or a polypeptide having an amino acid sequence selected from the group consisting of: SEQ ID NOs: 559-662. In some embodiments, the linker region comprises a polypeptide having at least 80% identity, at least 90% identity, at least 95% identity, or at least 97% identity to an amino acid sequence selected from the group consisting of: SEQ ID NOs: 153-202, or a polypeptide comprising an amino acid sequence selected from the group consisting of: SEQ ID NOs: 153-202. In other embodiments, the multi-domain defensin polypeptide comprises an amino acid sequence having at least 80% identity, at least 90% identity, at least 95% identity, or at least 97% identity to an amino acid sequence selected from the group consisting of: SEQ ID NOs: 52-102, 329-454, and 1156, or a polypeptide comprising an amino acid sequence selected from the group consisting of SEQ ID NOs: 52-102, 329-454 and 1156. In further embodiments, the first defensin region or the second defensin region of a multi-domain defensin comprises an amino acid sequence having at least 80% identity, at least 90% identity, at least 95% identity, or at least 97% identity to a defensin sequence portion of an amino acid sequence selected from the group consisting of: SEQ ID NOs: 1054-1152 and 1154, or an amino acid sequence selected from the group consisting of SEQ ID NOs: 1054-1152 and 1154. In further embodiments, the multi-domain defensin polypeptide encoded by nucleic acid sequence further comprises an N-terminal transit signal sequence having at least 80% identity, at least 90% identity, at least 95% identity, or at least 97% identity to an amino acid sequence selected form the group consisting of SEQ ID NOs: 809-954.

In certain embodiments, the defensin polypeptide comprises an amino acid sequence having at least 80% identity, at least 90% identity, at least 95% identity, or at least 97% identity to a defensin sequence portion of an amino acid sequence selected from the group consisting of: SEQ ID NOs: 1054-1152 and 1154, or an amino acid sequence selected from the group consisting of: SEQ ID NOs: 1054-1152 and 1154. In further embodiments, the multi-domain defensin polypeptide encoded by nucleic acid sequence further comprises an N-terminal transit signal sequence having at least 80% identity, at least 90% identity, at least 95% identity, or at least 97% identity to an amino acid sequence selected form the group consisting of: SEQ ID NOs: 809-954.

In another aspect, the invention provides a synthetic promoter as set forth in SEQ ID NO: 1158 to express single domain defensin or multi-domain defensin in plant cells.

In another aspect, the invention provides a plant, seed, plant tissue, plant part, or cell comprising a recombinant DNA construct provided herein or comprising the multi-domain defensin polypeptide encoded by a recombinant DNA construct provided herein. The plant, seed, plant tissue, plant part, or cell may exhibit tolerance or activity against at least one plant fungal pathogen species within one or more of the following genera of fungi: Fusarium, Collectotrichum, Stenocarpella, and/or Phakopsora. The plant, seed, plant tissue, plant part, or cell may exhibit tolerance or activity against one or more of the following fungal species: Fusarium graminearum, Fusarium verticilloides, Collectotrichum graminicola, Stenocarpella maydis, and/or Phakopsora pachyrhizi.

In another aspect, the invention provides a microorganism comprising a recombinant DNA construct provided herein, or a DNA molecule or vector comprising a recombinant DNA construct provided herein. The DNA molecule or vector may comprise a polynucleotide sequence having at least 70% identity, at least 85% identity, at least 90% identity, at least 95% identity, or at least 97% identity to a nucleotide sequence selected from the group consisting of: SEQ ID NOs: 1-51, 203-328, 955-1053 and 1155, or a polynucleotide sequence having a nucleotide sequence selected from the group consisting of: SEQ ID NOs: 1-51, 203-328, 955-1053, and 1155.

The invention further provides a method for conferring fungal pathogen tolerance or resistance to a plant, seed, cell, or plant part comprising expressing in said plant, seed, cell, or plant part the single domain defensin or the multi-domain defensin polypeptide encoded by the recombinant DNA construct disclosed herein. The invention further provides methods for expressing the recombinant DNA constructs of the invention in a plant cell or microorganism to produce a multi-domain defensin polypeptide, for example such that the multi-domain defensin polypeptide accumulates in a plant cell at a higher level relative to a single domain (1D) defensin control. The invention further provides a method for producing a transgenic plant with resistance or tolerance to a fungal pathogen comprising transforming a plant cell or tissue with the recombinant DNA molecule or vector provided herein, and regenerating a transgenic plant.

In another aspect, the invention provides a recombinant DNA construct comprising a nucleic acid sequence encoding a defensin having at least 80% identity to an amino acid sequence selected from the group consisting of SEQ ID NOs: 1054-1152, wherein the nucleic acid sequence encoding the defensin polypeptide is operably linked to a promoter functional in a plant cell. In certain embodiments, the defensin polypeptide has an amino acid sequence selected from the group consisting of SEQ ID NOs: 1054-1152. In further embodiments, the promoter comprises a nucleotide sequence as set for in SEQ ID NO: 1158.

The invention further provides a plant, seed, plant tissue, plant part, or cell comprising a recombinant DNA construct described herein. In some embodiments, the plant, seed, plant tissue, plant part, or cell has tolerance or activity against at least one plant fungal pathogen selected from the group consisting of Fusarium, Collectotrichum, Stenocarpella, and Phakopsora, for example at least one fungal species selected from the group consisting of Fusarium graminearum, Fusarium verticilloides, Collectotrichum graminicola, Stenocarpella maydis, and Phakopsora pachyrhizi.

In a further aspect, the invention provides methods of producing a transgenic plant with tolerance to a fungal pathogen comprising transforming a plant cell or tissue with the recombinant DNA molecule or vector described herein, and regenerating a transgenic plant.

### BRIEF DESCRIPTION OF THE SEQUENCES

SEQ ID NOs: 1-51: Nucleotide sequences of native multi-domain defensins.
SEQ ID NOs: 52-102: Polypeptide sequences of native multi-domain defensins.
SEQ ID NOs: 103-152: Nucleotide sequences of linker regions from multi-domain defensins.
SEQ ID NOs: 153-202: Polypeptide sequences of linker regions from multi-domain defensins.
SEQ ID NOs: 203-328: Nucleotide sequences of several synthetic multi-domain defensins.
SEQ ID NOs: 329-454: Polypeptide sequences of several synthetic multi-domain defensins.
SEQ ID NOs: 455-558: Nucleotide sequences of defensin regions from multi-domain defensins.
SEQ ID NOs: 559-662: Polypeptide sequences of defensin regions from multi-domain defensins.
SEQ ID NOs: 663-808: Nucleotide sequences of transit signals (TS) of multi-domain defensins.
SEQ ID NOs: 809-954: Polypeptide sequences of TS of multi-domain defensins.
SEQ ID NOs: 955-1053: Nucleotide sequences of 1D defensins.
SEQ ID NOs: 1054-1152: Polypeptide sequences of 1D defensins.
SEQ ID NO: 1153: Nucleotide sequence of MEDsa.AFPm1.
SEQ ID NO: 1154: Polypeptide sequence of MEDsa.AFPm1.
SEQ ID NO: 1155: Nucleotide sequence of a synthetic heterodimeric defensin.
SEQ ID NO: 1156: Polypeptide sequence of a synthetic heterodimeric defensin.
SEQ ID NO: 1157: Nucleotide sequence of codon optimized PINSY.AFP1.
SEQ ID NO: 1158: Nucleotide sequence of a synthetic regulatory element for controlling transgene expression.

### BRIEF DESCRIPTION OF THE DRAWINGS

FIG. 1 shows: (Top) a diagram depicting the domain configuration of two single domain (1D) defensins and four homodimeric synthetic two-domain (2D) defensins derived from the naturally occurring single domain defensins Coix22 and MtDef4. L1 and L2 represent different 2D linker regions described herein. The N-terminal transit signal (TS) sequences are also shown. (Bottom) a diagram depicting the domain configuration of a heterodimeric synthetic 2D defensin derived from Coix22 and AMAru.AFP10. L3 represent a 2D linker region described herein.

FIG. 2 shows: Relative RNA and protein expression levels of several homodimeric 2D defensins as compared to those levels of the corresponding 1D defensin.

### DETAILED DESCRIPTION

Plant diseases caused by fungal or other microbial pathogens can severely impact yield in crop plants, resulting in millions of tons of grain loss annually. Fungal plant diseases can result from a combination of several pathogens, and nearly every field of crop plants may experience fungal disease pressure to some extent. The development of effective methods of fungal control has been hindered by a lack of available agents with activity against different fungal pathogens, and/or agents which can be effectively combined with existing methods for fungal control. Yield loss due to fungal disease in agricultural plants therefore remains a significant problem.

The invention provides novel anti-microbial peptides (AMPs) comprising defensin or defensin-like proteins, including multi-domain defensin proteins, capable of conferring pest resistance or tolerance and/or fungicidal activity to plants. Novel polynucleotide molecules and sequences encoding defensin or defensin-like proteins, as well as recombinant DNA constructs comprising these novel defensin-encoding polynucleotide sequences, are also provided. In addition, methods are provided for producing plants with increased pest control or pesticidal activity by expressing in a plant a polynucleotide of the invention encoding a defensin or defensin-like protein, such as by transforming a plant or plant cell with a recombinant DNA construct comprising a polynucleotide sequence encoding a defensin or defensin-like protein, and transgenic plants or plant cells produced by these methods and comprising a defensin-encoding DNA construct of the invention. Pesticidal and plant health compositions and methods are also contemplated for administering or applying a defensin or defensin-like protein(s) of the invention to a plant, a plant growth medium or soil associated with the plant, or a plant part or seed.

The invention provides novel polynucleotide and polypeptide sequences of defensins or defensin-like protein molecules including multi-domain defensins, and the use of these sequences and molecules for generating pest resistance or tolerance in plants. As used herein, “tolerance” or “improved tolerance” in a plant to a pest or pathogen is an indication that the plant is less affected by the pest or pathogen with respect to yield, survivability and/or other relevant agronomic measures, compared to a less resistant, more “susceptible” plant. “Resistance” or “improved resistance” in a plant to a pest or pathogen is an indication that the plant is more able to reduce the effect of the pest or pathogen than a non-resistant or less resistant plant. The defensins described herein may be introduced into various plant species to confer anti-fungal and/or anti-microbial activity, and thus resistance or tolerance to one or more plant pests or pathogens. The defensins and defensin-like molecules disclosed herein may be introduced and expressed in a plant or plant cell to confer the anti-fungal and/or anti-microbial activity to the plant or plant cell. According to embodiments of the invention, polynucleotides, constructs and proteins of the invention may confer resistance or tolerance to, and activity against, one or more fungal pathogens, including a Fusarium, Colletotrichum, Stenocarpella, and/or Phakopsora species, such as Fusarium graminearum, Fusarium verticilloides, Colletotrichum graminicola, Stenocarpella maydis, and/or Phakopsora pachyrhizi. In addition to having anti-fungal activity, constructs and proteins of the invention may confer resistance or tolerance to, and activity against, other microbial plant pathogen(s) and/or plant pest(s), such as oomycetes, bacteria, insects, nematodes, etc.

Defensins or defensin-like proteins or polypeptides, hereinafter referred to jointly as defensins, are cysteine-rich cationic peptides, many of which exhibit inhibitory activity against a variety of microbial plant pathogens and agricultural pests. Single domain (1D) defensins are small globular proteins or peptide molecules, typically comprising approximately 50 amino acid (aa) residues that may be highly variable in sequence. Despite this variability, defensin peptides do share a gamma-thionin core consensus sequence (a gamma-thionin domain) and contain about 6-8 cysteine residues that may form disulfide bonds with protein folding. The three-dimensional structure of 1D defensins has been described as a Cys-stabilized αβ motif (CSαβ) having three anti-parallel β-sheets and one α-helix stabilized by multiple disulfide bridges formed by conserved cysteine residues (typically four disulfide bridges formed by eight conserved cysteine residues). See, e.g., Carvalho, A O et al., Peptides 30: 1007-1020 (2009); and Thomma, B et al., Planta 216: 193-202 (2002), the entire contents and disclosures of which are incorporated herein by reference. Defensins may further comprise an N-terminal transit signal (TS) sequence of variable length from only a few amino acids up to 20-30 amino acids, and/or a C-terminal extension sequence of variable length up to 35-30 amino acids when present. The N-terminal TS sequence on a defensin pro-protein may play a role in the targeting and/or export of the mature defensin protein into the apoplastic space or other sub-cellular compartment. However, the TS sequence may generally become cleaved and removed from the remainder of the defensin co/post-translationally to produce a mature defensin protein or peptide without the TS sequence. Some defensins may also have a C-terminal extension sequence that may also play a variety of roles in a plant cell and/or become cleaved from a mature defensin protein or peptide.

In one aspect, the invention provides several multi-domain (MD) defensins from plants, including two-domain (2D) and four-domain (4D) defensins, comprising two or more defensin regions or domains connected or bridged together by one or more linker regions, in addition to N-terminal TS sequences and/or C-terminal extension sequences. Each of the defensin regions of a 2D or MD defensin may also be referred to as a “defensin component” of the 2D or MD defensin. Thus, polypeptide sequences of the invention may comprise one of these multi-domain defensin proteins or polypeptides. Examples of multi-domain defensins including 2D defensins that may be used according to embodiments of the invention include those provided herein as SEQ ID NOs: 52-102. Further provided are polynucleotide molecules and constructs encoding one of these multi-domain defensins, such as those provided as SEQ ID NOs: 1-51. These MD defensin-encoding polynucleotides may be used according to embodiments of the invention to confer pesticidal and/or anti-fungal activity when expressed in a plant. Multi-domain defensin proteins of the invention may also optionally comprise an N-terminal transit signal (TS) sequence, such as one of SEQ ID NOs: 809-954, and/or a C-terminal sequence, such as one identified by annotation in Table 2 below for SEQ ID NOs: 101 and 102.

In one aspect, the invention provides several single domain (1D) defensins from plants that have pesticidal and/or anti-fungal activity, as well as polynucleotides encoding these 1D defensins. Thus, embodiments of the invention include polynucleotides and constructs encoding these 1D defensins for expression in a plant, or at least comprising a defensin sequence portion of these 1D defensins as identified herein. Examples of 1D defensins that are used according to embodiments of the invention include one or more of those provided herein as SEQ ID NOs: 1054-1127 and 1129-1152, and examples of polynucleotide sequences encoding these 1D defensins include one or more of those provided herein as SEQ ID NOs: 955-1028 and 1030-1053. 1D defensins of the invention may further include those comprising a defensin component of a 2D or MD defensin identified herein, such as one or more of those provided herein as SEQ ID NOs: 559-662, as well as polynucleotides sequences encoding these defensin components, such as one or more of those provided herein as SEQ ID NOs: 455-558. It is further contemplated that 1D defensins of the invention may comprise polypeptide and polynucleotide sequences having a relaxed sequence identity relative to the above identified sequences. For example, a 1D defensin may have a polypeptide sequence that is at least 80%, at least 85%, at least 90%, at least 95%, at least 97%, at least 98%, at least 99%, or 100% identical to at least a defensin sequence portion of one of SEQ ID NOs: 1054-1127 or 1129-1152, and/or a 1D defensin may be encoded by a polynucleotide sequence that is at least 80%, at least 85%, at least 90%, at least 95%, at least 97%, at least 98%, at least 99%, or 100% identical to at least a defensin sequence portion of one of SEQ ID NOs: 955-1028 or 1030-1053. A 1D defensin may further comprise a fragment comprising at least 25, least 50, at least 75, or at least 100, contiguous amino acids of a defensin sequence portion of one of SEQ ID NOs: 1054-1127 or 1129-1152, and/or a 1D defensin may be encoded by a polynucleotide sequence that is fragment comprising at least 25, at least 50, at least 75, or at least 100 contiguous nucleotides of a defensin sequence portion of one of SEQ ID NOs: 955-1028 or 1030-1053. In some embodiments, a fragment has the activity of the full-length defensin sequence portion of one of SEQ ID NOs: 1054-1127 or 1129-1152, or encoded by one of SEQ ID NOs: 955-1028 or 1030-1053. The 1D defensin-encoding polynucleotides and constructs may be used according to embodiments of the invention to confer pesticidal and/or anti-fungal activity when introduced and expressed in a plant.

In addition to newly discovered single and multi-domain defensins from plants, methods and compositions are provided for the construction, synthesis and expression of synthetic multi-domain defensin proteins comprising heterologous or non-naturally occurring combinations of two or more defensin regions or domains and one or more linker region(s) connecting or bridging the two or more defensin regions together, as well as polynucleotides encoding these synthetic multi-domain defensins. For example, each of the defensin regions of a synthetic multi-domain defensin may comprise a defensin sequence portion of a 1D defensin or a defensin component of a 2D or MD defensin, as identified herein. These synthetic multi-domain defensin proteins may thus be referred to as chimeric multi-domain defensins comprising defensin region(s) and/or linker region(s) that are heterologous in their origin. In addition to their novel structure, synthetic multi-domain defensins may also have one or more novel properties or characteristics, such as increased accumulation when expressed in a plant cell and/or new, altered or enhanced anti-fungal and/or pesticidal activity, relative to existing or known defensins, such as 1D defensins comprising one of their defensin components. A synthetic multi-domain defensin of the invention may comprise a heterologous or non-naturally occurring combination of a first defensin domain or region, a second defensin domain or region, and a linker region, wherein the first defensin region and the second defensin region are linked or connected to each other by the linker region. For example, a synthetic 2D defensin of the invention can comprise these regions in the following order (in the N-terminal to C-terminal direction): (i) first defensin region, (ii) linker region, and (iii) second defensin region. Synthetic multi-domain defensins may further comprise additional defensin region(s) connected by additional linker region(s). Furthermore, a 3D defensin may have the following order: (i) first defensin region, (ii) first linker region, (iii) second defensin region, (iv) second linker region, and (v) third defensin region, whereas a 4D defensin may additionally comprise a third linker region and a fourth defensin region with the third linker region being between the third and fourth defensin regions, and so on.

Accordingly, synthetic multi-domain defensin proteins or polypeptides of the invention may comprise combinations of two or more defensin domains or regions, each of the defensin region(s) comprising of one of the sequences provided herein as SEQ ID NOs: 559-662 and/or a defensin sequence portion of one of SEQ ID NOs: 1054-1152 and/or 1154, wherein the two or more defensin regions are linked or joined together by linker region(s), such as one or more of those provided herein as SEQ ID NOs: 153-202. For example, the first defensin region of a synthetic 2D defensin may comprise one of SEQ ID NOs: 559-662, and/or a defensin sequence portion of one of SEQ ID NOs: 1054-1152 and/or 1154, the second defensin region may comprise one of SEQ ID NOs: 559-662 and/or a defensin sequence portion of one of SEQ ID NOs: 1054-1152 and/or 1154, which may be the same as, or different than, the first defensin region, and the linker region connecting the first and second defensin regions may be one of SEQ ID NOs: 153-202.

In one embodiment of the invention, a “defensin sequence portion” shall refer to the sequence portion of a 1D defensin or defensin-like protein that excludes the N-terminal TS sequence and the C-terminal extension sequence (if present), as well as a polynucleotide sequence encoding the defensin sequence portion of a defensin or defensin-like protein. See, e.g., Table 15 below providing annotation for the defensin sequence portion of 1D defensin sequences. However, according to some embodiments, a defensin region near the N-terminus of a synthetic 2D or MD protein may retain its native TS sequence, and/or a defensin region near the C-terminus of the synthetic 2D or MD protein may retain its native C-terminal extension. Synthetic multi-domain defensins of the invention may also optionally comprise an N-terminal transit signal (TS) sequence, such as one of SEQ ID NOs: 809-954, and/or a C-terminal sequence, such as one identified by annotation in Table 2 below for SEQ ID NOs: 101 and 102. For example, synthetic multi-domain defensins of the invention may comprise one or more of those provided herein as SEQ ID NOs: 329-454 and 1156.

Polynucleotides of the invention may include sequences encoding synthetic multi-domain defensins. These polynucleotides may comprise combinations of two or more polynucleotide sequences encoding defensin domains or regions, each of these polynucleotide sequences encoding a defensin region may comprise a polynucleotide sequence provided herein as SEQ ID NOs: 455-558 or a defensin sequence portion of one of the polynucleotide sequences provided herein as SEQ ID NOs: 955-1053 and/or 1153, linked or joined together by a polynucleotide sequence(s) encoding a linker region(s), such as one or more of those provided herein as SEQ ID NOs: 103-152. For example, the sequence encoding the first defensin region of a synthetic 2D defensin may comprise one of SEQ ID NOs: 455-558 or a defensin sequence portion of one of the polynucleotide sequences provided herein as SEQ ID NOs: 955-1053 and/or 1153, the sequence encoding the second defensin region of the synthetic 2D defensin may comprise one of SEQ ID NOs: 455-558 or a defensin sequence portion of one of the polynucleotide sequences provided herein as SEQ ID NOs: 955-1053 and/or 1153, which may be the same as or different than the sequence encoding the first defensin region, and the sequence encoding the linker region of the synthetic 2D defensin connecting the first and second defensin regions may be one of SEQ ID NOs: 103-152. Polynucleotides of the invention may also optionally comprise sequences encoding a N-terminal targeting signal (TS) sequence, such as one of SEQ ID NOs: 663-808, and/or a C-terminal sequence, such as one of the sequences identified by annotation in Table 1 herein for SEQ ID NOs: 50 and 51. For example, polynucleotides encoding synthetic multi-domain defensins of the invention may comprise one or more of those provided herein as SEQ ID NOs: 203-328 and 1155.

Multi-domain defensin proteins of the invention may further include variants and homologues of the native and synthetic defensin sequences provided herein. According to some embodiments, multi-domain defensin variants may comprise one or more mutations, deletions, insertions, etc., relative to a native or synthetic defensin or multi-domain defensin sequence, or other engineered defensin-like sequences. Multi-domain defensin proteins or polypeptides of the invention may comprise combinations of two or more defensin domains or regions, wherein one or more of those defensin regions has a relaxed sequence identity relative to one or identity to one or more of SEQ ID NOs: 559-662, and/or a defensin sequence portion of one or more of SEQ ID NOs: 1054-1152 and/or 1154. Each defensin region of a multi-domain defensin may be at least 80% identical, at least 85% identical, at least 90% identical, at least 95% identical, at least 97% identical, at least 98% identical, at least 99% identical, or 100% identical to one of SEQ ID NOs: 559-662 and/or a defensin sequence portion of one of SEQ ID NOs: 1054-1152 and/or 1154. A defensin region of a multi-domain defensin may further comprise a fragment comprising at least 25, at least 50, at least 75, or at least 100, contiguous amino acids of one of SEQ ID NOs: 1054-1152 and/or 1154. In some embodiments, a fragment has the activity of the full-length defensin region of one of SEQ ID NOs: 1054-1154. Each of the linker domain(s) or region(s) of a multi-domain defensin protein may also have a relaxed sequence identity relative to SEQ ID NOs: 153-202. Each linker region of a multi-domain defensin may be at least 80% identical, at least 85% identical, at least 90% identical, at least 95% identical, at least 97% identical, at least 98% identical, at least 99% identical, or 100% identical to one of SEQ ID NOs: 153-202. Indeed, multi-domain defensin proteins of the invention may comprise a polypeptide sequence that is at least 80% identical, at least 85% identical, at least 90% identical, at least 95% identical, at least 97% identical, at least 98% identical, at least 99% identical, or 100% identical to any one of SEQ ID NOs: 52-102, 329-454, or 1156. A multi-domain defensin protein may further comprise a fragment comprising at least 50, at least 75, at least 100, at least 125, at least 150, at least 175, or at least 200 contiguous amino acids of any one of SEQ ID NOs: 52-102, 329-454 or 1156. In some embodiments, a fragment has the activity of SEQ ID NOs: 52-102, 329-454, or 1156. However, a linker region of a multi-domain may also be highly variable in sequence and length with little or no sequence identity of similarity to SEQ ID NOs: 153-202. A linker region may also comprise two or more linker sequences arranged in tandem, wherein each of the linker sequences may comprise one of SEQ ID NOs: 153-202, a polypeptide sequence having a relaxed sequence identity relative to SEQ ID NOs: 153-202, or other sequence.

The N-terminal targeting signal (TS) and/or a C-terminal sequences of a multi-domain defensin (if present) may each also have relaxed sequence identity relative to the sequences provided herein. The N-terminal targeting signal (TS) sequence may be at least 80% identical, at least 85% identical, at least 90% identical, at least 95% identical, at least 97% identical, at least 98% identical, at least 99% identical, or 100% identical to one of SEQ ID NOs: 809-954. The C-terminal sequence may be at least 80% identical, at least 85% identical, at least 90% identical, at least 95% identical, at least 97% identical, at least 98% identical, at least 99% identical, or 100% identical to one of the C-terminal sequences identified by annotation in Table 2 for SEQ ID NOs: 101 and 102. According to some embodiments, however, the N-terminal and/or C-terminal sequences of a multi-domain defensin may instead be dissimilar or have a lower sequence identity relative to the sequences provided herein.

Polynucleotides of the invention may comprise sequences encoding multi-domain defensin proteins with relaxed sequence identity relative to the sequences provided herein. Polynucleotides encoding these multi-domain defensins may comprise combinations of two or more sequences encoding defensin domains or regions, wherein one or more of the polynucleotide sequences encoding these defensin regions have a relaxed sequence identity relative to one or more of SEQ ID NOs: 455-558 and/or a defensin sequence portion of one or more of SEQ ID NOs: 955-1053 and/or 1153. Accordingly, polynucleotide sequences encoding each defensin region of a multi-domain defensin may be at least 70% identical, at least 75% identical, at least 80% identical, at least 85% identical, at least 90% identical, at least 95% identical, at least 97% identical, at least 98% identical, at least 99% identical, or 100% identical to one of SEQ ID NOs: 455-558 and/or a defensin sequence portion of one of SEQ ID NOs: 955-1053 and/or 1153. A defensin region of a multi-domain defensin may further comprise a fragment comprising at least 50, at least 75, at least 100, at least 125, at least 150, at least 175, or at least 200 contiguous nucleotides of any one of SEQ ID NOs: 455-558 and/or a defensin sequence portion of one of SEQ ID NOs: 955-1053 and/or 1153. In some embodiments, a fragment encodes a protein having the activity of a protein encoded by SEQ ID NOs: 455-558 and/or a defensin sequence portion of one of SEQ ID NOs: 955-1053 and/or 1153. The polynucleotide sequences encoding the linker domain(s) or region(s) of a multi-domain defensin protein may also have a relaxed sequence identity relative to SEQ ID NOs: 103-152. Accordingly, the polynucleotide sequence encoding a linker region of a multi-domain defensin may be at least 70% identical, at least 75% identical, at least 80% identical, at least 85% identical, at least 90% identical, at least 95% identical, at least 97% identical, at least 98% identical, at least 99% identical, or 100% identical to one of SEQ ID NOs: 103-152. Indeed, polynucleotides encoding multi-domain defensin proteins of the invention may comprise a polynucleotide sequence that is at least 70% identical, at least 75% identical, at least 80% identical, at least 85% identical, at least 90% identical, at least 95% identical, at least 97% identical, at least 98% identical, at least 99% identical, or 100% identical to any one of SEQ ID NOs: 1-51, 203-328, or 1155. However, a polynucleotide sequence encoding the linker region(s) of a multi-domain may also be highly variable in sequence and length with little or no sequence identity of similarity to SEQ ID NOs: 103-152. As mentioned above, a linker region may also comprise two or more linker sequences arranged in tandem, wherein each of the linker sequences may comprise one of SEQ ID NOs: 103-152, a polynucleotide sequence having a relaxed sequence identity relative to SEQ ID NOs: 103-152, or other sequence.

If present, polynucleotide sequences encoding the N-terminal targeting signal (TS) sequence and/or the C-terminal sequence of a multi-domain defensin may also have a relaxed sequence identity relative to sequences provided herein. The polynucleotide sequence encoding the N-terminal targeting signal (TS) sequence of a multi-domain defensin may be at least 70% identical, at least 75% identical, at least 80% identical, at least 85% identical, at least 90% identical, at least 95% identical, at least 97% identical, at least 98% identical, at least 99% identical, or 100% identical to one SEQ ID NOs: 663-808. The polynucleotide sequence encoding the C-terminal sequence of a multi-domain defensin may be at least 70% identical, at least 75% identical, at least 80% identical, at least 85% identical, at least 90% identical, at least 95% identical, at least 97% identical, at least 98% identical, at least 99% identical, or 100% identical to one of the C-terminal sequences identified in Table 1 for SEQ ID NOs: 50 and 51.

According to some embodiments, however, the N-terminal and/or C-terminal sequences of a multi-domain defensin may instead be dissimilar or have a lower sequence identity relative to the sequences provided herein.

For purposes of the invention, the percent identity of two polynucleotide or polypeptide sequences may be determined by first optimally aligning the two sequences and then determining the percentage of nucleotide bases or amino acid residues that are the same between the two sequences over a comparison window, which may be over the full length of one of the two sequences. An optimal alignment is defined as a best fit or match of the two sequences with resistance or tolerance for any gaps in the alignment. A number of computerized programs and algorithms are known in the art for achieving an optimal alignment of two or more sequences (GAP, BESTFIT, FASTA, BLAST, Smith-Waterman). When optimally aligned, the percent identity of a subject sequence to a reference sequence is determined by taking the number of matched or identical bases or amino acids between the two sequences, dividing by the length of the reference sequence, and then multiplying the quotient by 100%.

Multi-domain defensins of the invention may further have characteristic structural features, which may be related to their pesticidal and anti-fungal activity. These features may allow for the identification of additional defensins that may be used in designing novel multi-domain defensins. For example, multi-domain defensin proteins of the invention may be defined as comprising the following features: (a) a Pfam gamma-thionin domain (PF00304), as determined by a search of the Pfam protein families database using an E-value cutoff of 1e⁻³ (Finn, et al. Nucleic Acids Research, 2014, Database Issue 42:D222-D230); (b) polypeptides comprising more than 8 cysteine residues; and (c) polypeptides comprising two or more gamma-thionin domains (GXCXₙC) separated by a polypeptide linker sequence, wherein n is 3-22 amino acids in length, and X is any amino acid. These putative MD defensins may further have a protein length of at least 90 amino acids or greater and an absence of any premature stop codons in its coding sequence. Furthermore, each defensin region of a multi-domain defensin may be defined as having at least 6 cysteine residues, or at least 8 cysteine residues, and a predicted Cys-stabilized αβ motif (CSαβ) structure when folded as described above. However, one or more of the defensin region(s) may each have fewer cysteines according to some embodiments, such as 4 or 5 cysteines in one or more of the defensin region(s). Thus, a multi-domain defensin may have at least 8 cysteine residues, or at least 10 cysteine residues, at least 12 cysteine residues, or at least 14 cysteine residues, and/or two or more predicted CSαβ structural motifs. Multi-domain defensins may also be defined functionally as having certain pesticidal or anti-fungal activity against one or more plant fungal pathogens.

As introduced above, the invention provides synthetic 2D or other MD defensins comprising at least a first defensin region linked to a second defensin region by a linker region. The defensin regions of a multi-domain defensin protein may be homomeric (i.e., the defensin regions are the same) or heteromeric (i.e., the defensin regions are different). A 2D or MD defensin of the invention may comprise a native combination of defensin regions (i.e., comprising the same combination of defensin regions present in a native multi-domain defensin) or a heterologous combination of defensin regions (i.e., comprising defensin regions that do not exist together in a native multi-domain defensin and/or derived from different defensins). One or more of the defensin region(s) may be heterologous with respect to a linker region(s) of the MD defensin. Indeed, a multi-domain defensin of the disclosure may comprise a native combination of defensin regions that may be homomeric or heteromeric relative to each other but heterologous with respect to a linker region. Without being bound by any theory, synthetic multi-domain defensins having a linker region that is heterologous with respect to at least one defensin region may tend to accumulate to higher levels when expressed in a plant or plant cell and/or confer new or altered anti-fungal or anti-microbial activities.

As further described above, embodiments of the disclosure provide nucleic acids and polynucleotides comprising one or more of SEQ ID NOs: 455-558 and/or a defensin sequence portion of one or more of 955-1053, such as SEQ ID NOs: 1-51, 203-328, or 1155 and defensin polypeptides or proteins encoded by these polynucleotide sequences. Defensin polypeptides or proteins may comprise one or more of SEQ ID NOs: 559-662 and/or a defensin sequence portion of one or more of 1054-1152, such as SEQ ID NOs: 52-102, 329-454, or 1156. Complements to any of the nucleic acid or polynucleotide sequences described herein are also provided. Polynucleotide molecules of the invention may encode a multi-domain defensin polypeptide comprising two or more defensin domains or regions, such as a 2D, 3D, 4D, or other MD defensin protein. Each defensin region of a multi-domain defensin is separated from all other defensin regions by one or more linker region(s) with at least one linker region being present between adjacent or neighboring defensin regions. In specific embodiments, a multi-domain defensin protein may comprise two or more defensin domains each comprising 4, 5, 6, 7, 8, 9, 10, or more cysteine residues. In certain embodiments as explained above, a polynucleotide molecule provided herein may be defined as comprising one or more nucleotide sequences, each having at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to the full length sequence of SEQ ID NOs: 1-51, 103-152, 203-328, 455-558, 663-808, 1155 and/or a defensin sequence portion of 955-1053 and/or 1153. Similarly, a polypeptide molecule provided herein may be defined as comprising one or more protein sequences, each having at least 80%, at least 85%, at least 90%, at least 91%, at least 92%, at least 93%, at least 94%, at least 95%, at least 96%, at least 97%, at least 98%, at least 99%, or 100% sequence identity to the full length sequence of SEQ ID NOs: 52-102, 153-202, 329-454, 559-662, 809-954, 1156, and/or a defensin sequence portion of 1054-1152 and/or 1154.

Polynucleotide sequences of the disclosure may, when expressed in a plant, confer increased pesticidal or fungicidal activity and/or increased pest or fungal control to the plant. Indeed, polynucleotide constructs encoding multi-domain defensins of the disclosure may be used to generate transgenic plants with greater resistance or tolerance to one or more fungal pathogens. Such pesticidal or fungicidal activity may be effective against one or more of a Fusarium, Colletotrichum, Stenocarpella, and/or Phakopsora species, such as one or more of Fusarium graminearum, Fusarium verticilloides, Colletotrichum graminicola, Stenocarpella maydis, and/or Phakopsora pachyrhizi. Such pesticidal activity may be further effective against other microbial plant pathogen(s) and/or plant pest(s), such as oomycetes, bacteria, insects, nematodes, etc.

The invention also provides recombinant DNA constructs comprising the polynucleotide sequences described herein, as well as plants, plant cells and seeds transformed therewith. In one embodiment, such DNA constructs may be used in expressing nucleotide sequences encoding 1D, 2D, or other MD defensins in plants for the purposes of protecting the plant from plant pests, such as one or more fungi. In another embodiment, such constructs may be of use in generating transgenic or recombinant plants with increased or enhanced resistance or tolerance to plant fungal pathogens. Thus, embodiments of the invention further comprise transformation vectors comprising a defensin-encoding DNA construct of the invention or a portion of such defensin-encoding DNA construct. According to some embodiments, defensin-encoding DNA constructs and vectors may be used for generating 1D, 2D, or MD defensin transcripts and/or proteins in microorganisms such as bacteria or yeast.

A recombinant DNA construct, molecule or vector of the invention may comprise a polynucleotide expression cassette comprising a coding sequence that encodes a 1D defensin or multi-domain defensin protein, wherein the coding sequence is operably linked to a promoter that is functional in a plant cell. Alternatively, the defensin-encoding polynucleotide sequence may be operably linked to a promoter suitable for expression of a defensin protein in a microorganism. Any suitable promoter known in the art may be used to express a defensin coding sequence of the invention in a plant, such as a constitutive, tissue-specific, tissue-enhanced or tissue-preferred, developmental, inducible, disease inducible, etc., promoter. Further, the plant promoter operably linked to the defensin coding sequence may be native, homologous or heterologous relative to the plant species to be transformed with the defensin-encoding DNA construct, or alternatively, the promoter may be chimeric or synthetic. A synthetic nucleotide sequence may be a nucleotide sequence that is not known to occur in nature or that is not naturally occurring. Generally, such a synthetic nucleotide sequence will comprise at least one nucleotide difference when compared to any other naturally occurring nucleotide sequence. It is recognized that a gene-regulatory element of the invention comprises a synthetic nucleotide sequence. Preferably, the synthetic nucleotide sequence shares little or no extended homology to natural sequences. Extended homology in this context generally refers to 100% sequence identity extending beyond about 25 nucleotides of contiguous sequence. A synthetic gene-regulatory element of the invention comprises a synthetic nucleotide sequence.

The polynucleotide coding sequence for expressing the defensin protein may also be operatively linked to one or more additional regulatory element(s), such as an enhancer(s), leader, transcription start site (TSS), linker, 5′ and 3′ untranslated region(s), intron(s), polyadenylation signal, termination region or sequence, etc., that are suitable or necessary for regulating or allowing expression of the multi-domain defensin in a plant cell. Such additional regulatory element(s) may be optional and/or used to enhance or optimize expression of the defensin transgene or coding sequence. The term “operably linked” refers to a functional connection between the two sequences. A promoter or enhancer is “operably linked” to a transgene or coding sequence by affecting, causing, driving, promoting, etc., transcription and expression of the transgene or coding sequence.

According to embodiments of the invention, the term “recombinant” in reference to a DNA molecule, construct, vector, etc., refers to a DNA molecule or sequence that is not found in nature and/or is present in a context in which it is not found in nature, including a DNA molecule, construct, etc., comprising a combination of DNA sequences that would not naturally occur contiguously or in close proximity together without human intervention, and/or a DNA molecule, construct, etc., comprising at least two DNA sequences that are heterologous with respect to each other. A recombinant DNA molecule, construct, etc., may comprise DNA sequence(s) that is/are separated from other polynucleotide sequence(s) that exist in proximity to such DNA sequence(s) in nature, and/or a DNA sequence that is adjacent to (or contiguous with) other polynucleotide sequence(s) that are not naturally in proximity with each other. A recombinant DNA molecule, construct, etc., may also refer to a DNA molecule or sequence that has been genetically engineered and constructed outside of a cell. For example, a recombinant DNA molecule may comprise any suitable plasmid, vector, etc., and may include a linear or circular DNA molecule. Such plasmids, vectors, etc., may contain various maintenance elements including a prokaryotic origin of replication and selectable marker, as well as a defensin expressing transgene or cassette perhaps in addition to a plant selectable marker gene, etc. The term “recombinant” may also further refer to proteins expressed or encoded by these recombinant DNA molecules, constructs, etc., if they comprise non-native sequences.

As used herein, the term “isolated” in reference to a DNA molecule, construct, vector, etc., may refer to a DNA molecule or sequence that is not found in nature and/or is present in a context in which it is not found in nature. The term “isolated” may also refer to a nucleic acid molecule that has undergone at least one step towards being isolated or concentrated or enriched from a more complex solution or source. The term “isolated,” however, is in no way intended to limit the nucleic acid molecule to a particular location or state, and the invention extends to the nucleic acid molecule when introduced into the genome of a cell or when it is resident in progeny of cells into which the nucleic acid molecule has been introduced into its genome.

According to another broad aspect of the invention, methods are provided for transforming a plant cell, tissue or explant with a recombinant DNA molecule or construct comprising a defensin transgene or coding sequence to produce a defensin containing transgenic plant. Numerous methods for transforming chromosomes in a plant cell with a recombinant DNA molecule are known in the art, which may be used according to methods of the invention to produce a transgenic plant cell and plant. Any suitable method or technique for transformation of a plant cell known in the art may be used according to present methods. Effective methods for transformation of plants include bacterially mediated transformation, such as Agrobacterium-mediated or Rhizhobium-mediated transformation, and microprojectile bombardment-mediated transformation. Other methods for plant transformation are also known in the art including, but not limited to, gene editing, site-directed integration, PEG-mediated transformation, protoplast transformation, electroporation, microinjection, agitation with silica/carbon fibers, virus-mediated or liposome-mediated transformation, etc. A variety of methods are known in the art for transforming explants with a transformation vector via bacterially mediated transformation or microprojectile bombardment and then subsequently culturing, etc, those explants to regenerate or develop transgenic plants. Methods are further provided for expressing a multi-domain defensin transgene of the invention in one or more plant cells or tissues under the control of a promoter operable in a plant cell. Such methods may be used to confer anti-fungal and pesticidal properties to a plant and combat a fungal infection.

Transformation of a target plant material or explant may be practiced in tissue culture on nutrient media, for example a mixture of nutrients that allow cells to grow in vitro. Recipient cell targets or explants may include, but are not limited to, meristems, shoot tips, protoplasts, hypocotyls, calli, immature or mature embryos, shoots, buds, nodal sections, leaves, gametic cells such as microspores, pollen, sperm and egg cells, etc., or any suitable portions thereof. It is contemplated that any transformable cell or tissue from which a fertile plant can be regenerated or grown/developed may be used as a target for transformation. Transformed explants, cells or tissues may be subjected to additional culturing steps, such as callus induction, selection, regeneration, etc., as known in the art. Transformed cells, tissues or explants containing a recombinant DNA insertion may be grown, developed or regenerated into transgenic plants in culture, plugs or soil according to methods known in the art. Transgenic plants may be further crossed to themselves or other plants to produce transgenic seeds and progeny. A transgenic plant may also be prepared by crossing a first plant comprising the recombinant DNA sequence or transformation event with a second plant lacking the insertion. For example, a recombinant DNA sequence may be introduced into a first plant line that is amenable to transformation, which may then be crossed with a second plant line to introgress the recombinant DNA sequence into the second plant line. Progeny of these crosses can be further back crossed into the more desirable line multiple times, such as through 6 to 8 generations or back crosses, to produce a progeny plant with substantially the same genotype as the original parental line but for the introduction of the recombinant DNA sequence.

A recombinant DNA molecule or construct of the invention may be included within a DNA transformation vector for use in transformation of a target plant cell, tissue or explant. Such a transformation vector of the invention may generally comprise sequences or elements necessary or beneficial for effective transformation in addition to the defensin expressing transgene or expression cassette. For Agrobacterium-mediated transformation, the transformation vector may comprise an engineered transfer DNA (or T-DNA) segment or region having two border sequences, a left border (LB) and a right border (RB), flanking at least the defensin expressing transgene or cassette, such that insertion of the T-DNA into the plant genome will create a transformation event for the defensin transgene or cassette. In other words, the defensin transgene would be located between the left and right borders of the T-DNA, perhaps along with an additional transgene(s) or expression cassette(s), such as a plant selectable marker transgene and/or other gene(s) of agronomic interest that may confer a trait or phenotype of agronomic interest to a plant. In addition to protein encoding sequences, a gene of agronomic interest may further comprise a polynucleotide sequence encoding a RNA suppression element. According to alternative embodiments, the defensin-encoding transgene or cassette and the plant selectable marker transgene (or other gene of agronomic interest) may be present in separate T-DNA segments on the same or different recombinant DNA molecule(s), such as for co-transformation. A transformation vector or construct may further comprise prokaryotic maintenance elements, which for Agrobacterium-mediated transformation may be located in the vector backbone outside of the T-DNA region(s).

A plant selectable marker transgene in a transformation vector or construct of the invention may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptII), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (aroA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known.

Additionally provided herein are transgenic plants, plant parts, propagules and plant cells transformed with a recombinant DNA construct or vector of the invention having a polynucleotide sequence encoding a multi-domain defensin. Such a transgenic plant, plant part, or plant cell may comprise a transformation event or insertion of a defensin-encoding recombinant DNA construct or sequence of the invention into the genome of at least one plant cell thereof. A transgenic plant comprising the defensin-encoding DNA construct or sequence may be produced by any suitable transformation method, which may be followed by selection, culturing, regeneration, development, etc., as desired or needed to produce a transgenic R₀ plant, which may then be selfed or crossed to other plants to generate R1 seed and subsequent progeny generations and seed through additional crosses, etc. Similarly, embodiments of the invention further include a plant cell, tissue, explant, etc., comprising one or more transgenic cells having a transformation event or genomic insertion of a recombinant DNA or polynucleotide sequence comprising the defensin-encoding transgene, construct or sequence. Transgenic plants comprising a defensin-encoding transgene may have increased resistance or tolerance to one or more plant pests or fungi and/or increased anti-fungal properties or activities, relative to a wild type or control plant not having the defensin-encoding transgene.

For purposes of the invention, a “plant” may include an explant, embryo, seedling, plantlet or whole plant at any stage of regeneration or development. As used herein, a “transgenic plant” refers to a plant whose genome has been altered by the integration or insertion of a recombinant DNA molecule, construct or sequence. A transgenic plant includes an R₀ plant developed or regenerated from an originally transformed plant cell(s) as well as progeny transgenic plants in later generations or crosses from the R₀ transgenic plant. As used herein, a “plant part” may refer to any organ or intact tissue of a plant, such as a meristem, shoot organ/structure (e.g., leaf, stem and tuber), root, flower or floral organ/structure (e.g., bract, sepal, petal, stamen, carpel, anther, pollen and ovule), seed (e.g., embryo, endosperm, and seed coat), fruit (e.g., the mature ovary), propagule, or other plant tissues (e.g., cuttings, vascular tissue, ground tissue, and the like), callus, protoplasts, or any portion thereof. Plant parts of the invention may be viable, nonviable, regenerable, and/or non-regenerable. A “propagule” may include any plant part that is capable of growing into an entire plant. As used herein, a “transgenic plant cell” simply refers to any plant cell that is transformed with a stably-integrated recombinant DNA molecule or sequence. A transgenic plant cell may include an originally-transformed plant cell, a transgenic plant cell of a regenerated or developed R₀ plant, or a transgenic plant cell from any progeny plant or offspring of the transformed R₀ plant, including cell(s) of a plant seed or embryo, or a cultured plant or callus cell, etc.

The transformed plants may be analyzed for the presence of the defensin-encoding sequence or transgene and/or its expression level and/or profile in a plant or plant cell or tissue. Those of skill in the art are aware of numerous methods available for the analysis of transformed plants. For example, methods for plant analysis include, but are not limited to, Southern blots or northern blots, PCR-based approaches, biochemical analyses, phenotypic screening methods, and immunoblotting assays. The expression of a transcribable DNA molecule or transgene can be measured, for example, using TaqMan® (Applied Biosystems, Foster City, Calif.) reagents and methods as described by the manufacturer and PCR cycle times determined using the TaqMan® Testing Matrix. As an alternative example, the Invader® (Third Wave Technologies, Madison, Wis.) reagents and methods as described by the manufacturer can be used to evaluate transgene expression.

The transgenic plants of the invention comprising a defensin-encoding polynucleotide sequence or construct can be any agricultural crop species. The species may be a monocotyledonous or dicotyledonous plant. Particularly useful plants may include but are not limited to wheat, carrot, sorghum rice, barley, soybean, potato, corn, Brassica, canola, tomato, alfalfa, peanut, sugarcane and cotton. The plant can be an R₀ transgenic plant (i.e., a plant derived from the original transformed tissue). The transgenic plant can also be any generation of progeny plants derived from the original R₀ transgenic plant, such as by any known method of crossing, introgressing, converting or propagating plants.

The invention also provides methods for producing a transgenic plant with increased pest resistance or tolerance and/or pesticidal activity comprising introducing or transforming into the plant a recombinant DNA construct encoding a defensin protein as described herein. Such a method may further comprise: growing said plant to produce a further generation; and selecting at least one plant from said further generation comprising the recombinant DNA construct, wherein said plant has increased pest resistance or tolerance and/or anti-fungal activity relative to a control plant that does not comprise the recombinant DNA construct. Further provided is a method for producing plants with increased pest resistance or tolerance and/or pesticidal activity comprising crossing a transgenic plant of the invention with itself or a second plant to produce at least a first progeny plant, wherein said progeny plant comprises increased pest resistance or tolerance. Seed from plants comprising the recombinant DNA construct and having increased pesticidal activity and/or pest resistance or tolerance may be obtained from any number of sources.

According to another aspect of the invention, pesticidal compositions are provided comprising a defensin polypeptide or protein of the invention having pesticidal or anti-fungal activity. Such pesticidal compositions may further comprise other compounds or active pesticidal molecules that may be effective against one or more insects, nematodes, microbes, fungi, nematodes, or viruses. These pesticidal compositions comprising one or more recombinant defensin(s) of the invention may be formulated as a solid or liquid and/or applied as a topical, foliar, soil, or granular application or treatment to prevent or inhibit fungal infections and/or other plant pest infestations. Examples of other types of plant pests include insects, nematodes, weeds, microbes, such as bacteria, fungi, and viruses, etc. Methods for formulating a pesticidal composition of the invention may be similar to methods known in the art for other pesticidal formulations. Ingredients or components for pesticidal composition of the invention may include one or more carriers, diluents, surfactants, or other formulation ingredients known in the art.

### EXAMPLES

### Example 1. Identification of Defensins with Two or More Defensin Regions

Plant defensins are small polypeptides comprising an N-terminal signal peptide and a defensin region comprising approximately 50 amino acids and usually having 6 to 8 cysteines. The invention provides new multi-domain defensins comprising two defensin regions (2D defensins) or multiple defensin regions (MD defensins) connected by a short linker region. Many of these 2D or MD defensins were further found to comprise an N-terminal transit signal (TS) sequence or region that may generally become cleaved from the remainder of the protein to produce a mature defensin protein. Some of the 2D or MD defensins were also found to comprise a C-terminal extension sequence.

To identify novel multi-domain defensins, genomic and transciptome sequences from 19 different plant species were mined and analyzed to identify longer polypeptide sequences having one or more structural or functional features characteristic of defensins. Polypeptides meeting the following criteria were identified as multi-domain defensins and subjected to further analysis: (a) Polypeptides comprising a Pfam gamma-thionin domain (PF00304), as determined by a search of the Pfam protein families database using an E-value cutoff of 1e⁻³ (Finn, et al. Nucleic Acids Research, 2014, Database Issue 42:D222-D230); (b) Polypeptides comprising at least 100 amino acids; (c) Polypeptides comprising at least 8 cysteine residues; and (d) Polypeptides comprising two or more extended gamma-thionin domains (GXCXₙC) separated by a short polypeptide linker sequence, wherein the n is 3-22 amino acids in length, and X is any amino acid. These putative multi-domain defensins were further identified by the absence of a premature stop codon in their coding sequence.

A total of 51 multi-domain defensin proteins including mostly 2D defensins were identified (see below) by mining the genomes of the following 19 plant genomes: Arabidopsis lyrata, Arabidopsis thaliana, Brassica rapa, Brassica napus, Carica papaya, Citrus clementina, Citrus sinensis, Cucumis melo, Cucumis sativus, Glycine max, Kochia scoparia, Malus domestica, Medicago truncatula, Portulaca oleracea, Prunus persica, Raphanus raphanistrum, Rosa blanda, Trifolium repens, and Zea mays. The detection of mRNA transcripts for these multi-domain defensins and their discovery in many plant species supports the conclusion that these multi-domain defensins are expressed in plants.

### Example 2. Annotation of Multi-Domain Defensins

Full-length multi-domain protein sequences identified by the above criteria were further analyzed using a combination of manual calls, multiple sequence alignments, and N-terminal cleavage site (SignalP) predictions to identify and locate the N-terminal TS or signal peptide sequences, defensin regions, intervening linker regions, and C-terminal extensions. Linker regions were identified as being between the last cysteine residue of the defensin region on the N-terminal side and one or two amino acids upstream of the first cysteine residue in the defensin region on the C-terminal side. Determining the position of the C-terminal end of the linker region of an identified 2D or MD defensin is based on the expected position of the predicted cleavage site of the downstream defensin region on the C-terminal side of the linker region, although this site may generally not become cleaved, unlike the cleavage site between the N-terminal TS sequence and the adjacent defensin region.

The 51 multi-domain defensin sequences identified from the 19 plant genomes are shown in Tables 1 and 2. SEQ ID NOs: 1-51 represent nucleotide coding sequences of the newly identified 2D or MD defensins, and SEQ ID NOs: 52-102 represent the corresponding polypeptide sequences of these identified 2D or MD defensins. For each of the identified 2D and MD defensins, the sequence boundaries (start and stop positions) for each of the defensin regions (D1, D2, etc.), linker region(s) (L1, L2, etc.), N-terminal transit signal (TS) sequence, and C-terminal (CT) extension sequence (if present; “NA” if not present) are shown for the DNA and protein sequences in Tables 1 and 2, respectively. The full coding sequence (CDS) is also provided in Table 1. The defensin components of these 2D or MD defensins correspond to each of the defensin regions annotated in Tables 1 and 2. For illustration, the defensin components of “ARAly_AFP26” on a nucleotide level correspond to nucleotides positions 82-207 (D1) and 229-372 (D2) of SEQ ID NO: 1, whereas the defensin components of “ARAly_AFP26” on a protein level correspond to amino acid positions 28-69 (D1) and 77-124 (D2) of SEQ ID NO: 52.

TABLE 1
Nucleotide start and end positions for each region within the identified MD or 2D defensin sequences.

| | | | TS | | CDS | | CT | | D1 | | D2 | |
|---|---|---|---|---|---|---|---|---|---|---|---|---|
| | DNA | Protein | TS | TS | CDS | CDS | CT | CT | D1 | D1 | D2 | D2 |
| Locus ID | SEQ ID NO. | SEQ ID NO. | Start | End | Start | End | Start | End | Start | End | Start | End |
| ARAly_AFP26 | 1 | 52 | 1 | 81 | 82 | 372 | NA | NA | 82 | 207 | 229 | 372 |
| At_AFP10 | 2 | 53 | 1 | 63 | 64 | 387 | NA | NA | 64 | 234 | 259 | 387 |
| At_AFP11 | 3 | 54 | 1 | 78 | 79 | 381 | NA | NA | 79 | 210 | 241 | 381 |
| At_AFP12 | 4 | 55 | 1 | 81 | 82 | 360 | NA | NA | 82 | 195 | 220 | 360 |
| At_AFP8 | 5 | 56 | 1 | 78 | 79 | 378 | NA | NA | 79 | 204 | 259 | 378 |
| At_AFP9 | 6 | 57 | 1 | 78 | 79 | 366 | NA | NA | 79 | 210 | 226 | 366 |
| BASsc_AFP21 | 7 | 58 | 1 | 21 | 22 | 354 | NA | NA | 22 | 162 | 193 | 354 |
| Bn_AFP35 | 8 | 59 | 1 | 99 | 100 | 402 | NA | NA | 100 | 228 | 259 | 402 |
| Bn_AFP36 | 9 | 60 | 1 | 18 | 19 | 330 | NA | NA | 19 | 150 | 187 | 330 |
| Bn_AFP47 | 10 | 61 | 1 | 78 | 79 | 366 | NA | NA | 79 | 204 | 238 | 366 |
| Bn_AFP74 | 11 | 62 | 1 | 78 | 79 | 381 | NA | NA | 79 | 210 | 238 | 381 |
| Bn_AFP75 | 12 | 63 | 1 | 45 | 46 | 357 | NA | NA | 46 | 180 | 214 | 357 |
| Bn_AFP76 | 13 | 64 | 1 | 78 | 79 | 381 | NA | NA | 79 | 210 | 238 | 381 |
| Bn_AFP79 | 14 | 65 | 1 | 78 | 79 | 366 | NA | NA | 79 | 192 | 223 | 366 |
| Bn_AFP80 | 15 | 66 | 1 | 78 | 79 | 366 | NA | NA | 79 | 192 | 223 | 366 |
| Br_AFP1 | 16 | 67 | 1 | 42 | 43 | 333 | NA | NA | 43 | 177 | 241 | 333 |
| Br_AFP2 | 17 | 68 | 1 | 78 | 79 | 360 | NA | NA | 79 | 198 | 220 | 360 |
| CARpa_AFP1 | 18 | 69 | 1 | 45 | 46 | 402 | NA | NA | 46 | 192 | 217 | 402 |
| CARpa_AFP2 | 19 | 70 | 1 | 63 | 64 | 420 | NA | NA | 64 | 207 | 232 | 420 |
| CARpa_AFP3 | 20 | 71 | 1 | 69 | 70 | 384 | NA | NA | 70 | 192 | 202 | 384 |
| CITcl_AFP10 | 21 | 72 | 1 | 81 | 82 | 393 | NA | NA | 82 | 198 | 208 | 393 |
| CITcl_AFP11 | 22 | 73 | 1 | 66 | 67 | 402 | NA | NA | 67 | 195 | 220 | 402 |
| CITcl_AFP2 | 23 | 74 | 1 | 90 | 91 | 408 | NA | NA | 91 | 225 | 247 | 408 |
| CITcl_AFP3 | 24 | 75 | 1 | 75 | 76 | 390 | NA | NA | 76 | 216 | 250 | 390 |
| CITsi_AFP1 | 25 | 76 | 1 | 93 | 94 | 411 | NA | NA | 94 | 216 | 226 | 411 |
| CITsi_AFP2 | 26 | 77 | 1 | 54 | 55 | 369 | NA | NA | 55 | 195 | 226 | 369 |
| CUCme_AFP1 | 27 | 78 | 1 | 24 | 25 | 345 | NA | NA | 25 | 168 | 205 | 345 |
| CUCsa_AFP3 | 28 | 79 | 1 | 24 | 25 | 345 | NA | NA | 25 | 168 | 205 | 345 |
| Gm_AFP1 | 29 | 80 | 1 | 81 | 82 | 411 | NA | NA | 82 | 207 | 217 | 411 |
| Gm_AFP2 | 30 | 81 | 1 | 81 | 82 | 387 | NA | NA | 82 | 207 | 244 | 387 |
| MALdo_AFP11 | 31 | 82 | 1 | 87 | 88 | 408 | NA | NA | 88 | 219 | 250 | 408 |
| Mt_AFP14 | 32 | 83 | 1 | 87 | 88 | 408 | NA | NA | 88 | 237 | 259 | 408 |
| Mt_AFP60 | 33 | 84 | 1 | 84 | 85 | 390 | NA | NA | 85 | 204 | 244 | 390 |
| Mt_AFP65 | 34 | 85 | 1 | 78 | 79 | 384 | NA | NA | 79 | 204 | 235 | 384 |
| Mt_AFP66 | 35 | 86 | 1 | 69 | 70 | 285 | NA | NA | 70 | 105 | 142 | 285 |
| Mt_AFP67 | 36 | 87 | 1 | 81 | 82 | 387 | NA | NA | 79 | 207 | 235 | 387 |
| Mt_AFP77 | 37 | 88 | 1 | 81 | 82 | 390 | NA | NA | 82 | 189 | 232 | 390 |
| Mt_AFP78 | 38 | 89 | 1 | 78 | 79 | 384 | NA | NA | 79 | 207 | 238 | 384 |
| PORol_AFP1 | 39 | 90 | 1 | 63 | 64 | 360 | NA | NA | 64 | 228 | 256 | 360 |
| PORol_AFP2 | 40 | 91 | NA | NA | 1 | 297 | NA | NA | 1 | 165 | 193 | 297 |
| PORol_AFP27 | 41 | 92 | 1 | 87 | 88 | 390 | NA | NA | 88 | 219 | 241 | 390 |
| PRUpe_AFP1 | 42 | 93 | 1 | 18 | 19 | 402 | NA | NA | 19 | 201 | 211 | 402 |
| RAPra_AFP23 | 43 | 94 | 1 | 96 | 97 | 411 | NA | NA | 97 | 228 | 265 | 411 |
| RAPra_AFP24 | 44 | 95 | 1 | 78 | 79 | 384 | NA | NA | 79 | 213 | 238 | 384 |
| RAPra_AFP25 | 45 | 96 | 1 | 27 | 28 | 345 | NA | NA | 28 | 165 | 199 | 345 |
| RAPra_AFP26 | 46 | 97 | 1 | 9 | 10 | 294 | NA | NA | 10 | 132 | 163 | 294 |
| RAPra_AFP27 | 47 | 98 | 1 | 78 | 79 | 363 | NA | NA | 79 | 201 | 232 | 363 |
| ROSbl_AFP11 | 48 | 99 | 1 | 78 | 79 | 360 | NA | NA | 79 | 204 | 214 | 360 |
| TRIre_AFP5 | 49 | 100 | 1 | 84 | 85 | 387 | NA | NA | 85 | 204 | 241 | 387 |
| Zm_AFP101 | 50 | 101 | 1 | 24 | 25 | 420 | 421 | 474 | 25 | 120 | 283 | 420 |
| Zm_AFP100 | 51 | 102 | 1 | 162 | 163 | 966 | 967 | 1002 | 163 | 312 | 397 | 534 |

| | D3 | | D4 | | L1 | | L2 | | L3 | |
|---|---|---|---|---|---|---|---|---|---|---|
| | D3 | D3 | D4 | D4 | Linker1 | Linker1 | Linker2 | Linker2 | Linker3 | Linker3 |
| Locus ID | Start | End | Start | End | Start | End | Start | End | Start | End |
| ARAly_AFP26 | | | | | 208 | 228 | | | | |
| At_AFP10 | | | | | 235 | 258 | | | | |
| At_AFP11 | | | | | 211 | 240 | | | | |
| At_AFP12 | | | | | 196 | 219 | | | | |
| At_AFP8 | | | | | 205 | 258 | | | | |
| At_AFP9 | | | | | 211 | 225 | | | | |
| BASsc_AFP21 | | | | | 163 | 192 | | | | |
| Bn_AFP35 | | | | | 229 | 258 | | | | |
| Bn_AFP36 | | | | | 151 | 186 | | | | |
| Bn_AFP47 | | | | | 205 | 237 | | | | |
| Bn_AFP74 | | | | | 211 | 237 | | | | |
| Bn_AFP75 | | | | | 181 | 213 | | | | |
| Bn_AFP76 | | | | | 211 | 237 | | | | |
| Bn_AFP79 | | | | | 193 | 222 | | | | |
| Bn_AFP80 | | | | | 193 | 222 | | | | |
| Br_AFP1 | | | | | 178 | 240 | | | | |
| Br_AFP2 | | | | | 199 | 219 | | | | |
| CARpa_AFP1 | | | | | 193 | 216 | | | | |
| CARpa_AFP2 | | | | | 208 | 231 | | | | |
| CARpa_AFP3 | | | | | 193 | 201 | | | | |
| CITcl_AFP10 | | | | | 199 | 207 | | | | |
| CITcl_AFP11 | | | | | 196 | 219 | | | | |
| CITcl_AFP2 | | | | | 226 | 246 | | | | |
| CITcl_AFP3 | | | | | 217 | 249 | | | | |
| CITsi_AFP1 | | | | | 217 | 225 | | | | |
| CITsi_AFP2 | | | | | 196 | 225 | | | | |
| CUCme_AFP1 | | | | | 169 | 204 | | | | |
| CUCsa_AFP3 | | | | | 169 | 204 | | | | |
| Gm_AFP1 | | | | | 208 | 216 | | | | |
| Gm_AFP2 | | | | | 208 | 243 | | | | |
| MALdo_AFP11 | | | | | 220 | 249 | | | | |
| Mt_AFP14 | | | | | 238 | 258 | | | | |
| Mt_AFP60 | | | | | 205 | 243 | | | | |
| Mt_AFP65 | | | | | 205 | 234 | | | | |
| Mt_AFP66 | | | | | 106 | 141 | | | | |
| Mt_AFP67 | | | | | 208 | 234 | | | | |
| Mt_AFP77 | | | | | 190 | 231 | | | | |
| Mt_AFP78 | | | | | 208 | 237 | | | | |
| PORol_AFP1 | | | | | 229 | 255 | | | | |
| PORol_AFP2 | | | | | 166 | 192 | | | | |
| PORol_AFP27 | | | | | 220 | 240 | | | | |
| PRUpe_AFP1 | | | | | 202 | 210 | | | | |
| RAPra_AFP23 | | | | | 229 | 264 | | | | |
| RAPra_AFP24 | | | | | 214 | 237 | | | | |
| RAPra_AFP25 | | | | | 166 | 198 | | | | |
| RAPra_AFP26 | | | | | 133 | 162 | | | | |
| RAPra_AFP27 | | | | | 202 | 231 | | | | |
| ROSbl_AFP11 | | | | | 205 | 213 | | | | |
| TRIre_AFP5 | | | | | 205 | 240 | | | | |
| Zm_AFP101 | | | | | 121 | 282 | | | | |
| Zm_AFP100 | 613 | 750 | 829 | 966 | 313 | 396 | 535 | 612 | 751 | 828 |

TABLE 2
Protein start and end positions for each region within the identified MD or 2D defensin sequences.

| | | | TS | | CT | | D1 | | D2 | |
|---|---|---|---|---|---|---|---|---|---|---|
| | DNA | Protein | TS | TS | CT | CT | D1 | D1 | D2 | D2 |
| Locus ID | SEQ ID NO. | SEQ ID NO. | Start | End | Start | End | Start | End | Start | End |
| ARAly_AFP26 | 1 | 52 | 1 | 27 | NA | NA | 28 | 69 | 77 | 124 |
| At_AFP10 | 2 | 53 | 1 | 21 | NA | NA | 22 | 78 | 87 | 129 |
| At_AFP11 | 3 | 54 | 1 | 26 | NA | NA | 27 | 70 | 81 | 127 |
| At_AFP12 | 4 | 55 | 1 | 27 | NA | NA | 28 | 65 | 74 | 120 |
| At_AFP8 | 5 | 56 | 1 | 26 | NA | NA | 27 | 68 | 87 | 126 |
| At_AFP9 | 6 | 57 | 1 | 26 | NA | NA | 27 | 70 | 76 | 122 |
| BASsc_AFP21 | 7 | 58 | 1 | 7 | NA | NA | 8 | 54 | 65 | 118 |
| Bn_AFP35 | 8 | 59 | 1 | 33 | NA | NA | 34 | 76 | 87 | 134 |
| Bn_AFP36 | 9 | 60 | 1 | 6 | NA | NA | 7 | 50 | 63 | 110 |
| Bn_AFP47 | 10 | 61 | 1 | 26 | NA | NA | 27 | 68 | 80 | 122 |
| Bn_AFP74 | 11 | 62 | 1 | 26 | NA | NA | 27 | 70 | 80 | 127 |
| Bn_AFP75 | 12 | 63 | 1 | 15 | NA | NA | 16 | 60 | 72 | 119 |
| Bn_AFP76 | 13 | 64 | 1 | 26 | NA | NA | 27 | 70 | 80 | 127 |
| Bn_AFP79 | 14 | 65 | 1 | 26 | NA | NA | 27 | 64 | 75 | 122 |
| Bn_AFP80 | 15 | 66 | 1 | 26 | NA | NA | 27 | 64 | 75 | 122 |
| Br_AFP1 | 16 | 67 | 1 | 14 | NA | NA | 15 | 59 | 81 | 111 |
| Br_AFP2 | 17 | 68 | 1 | 26 | NA | NA | 27 | 66 | 74 | 120 |
| CARpa_AFP1 | 18 | 69 | 1 | 15 | NA | NA | 16 | 64 | 73 | 134 |
| CARpa_AFP2 | 19 | 70 | 1 | 21 | NA | NA | 22 | 69 | 78 | 140 |
| CARpa_AFP3 | 20 | 71 | 1 | 23 | NA | NA | 24 | 64 | 68 | 128 |
| CITcl_AFP10 | 21 | 72 | 1 | 27 | NA | NA | 28 | 66 | 70 | 131 |
| CITcl_AFP11 | 22 | 73 | 1 | 22 | NA | NA | 23 | 65 | 74 | 134 |
| CITcl_AFP2 | 23 | 74 | 1 | 30 | NA | NA | 31 | 75 | 83 | 136 |
| CITcl_AFP3 | 24 | 75 | 1 | 25 | NA | NA | 26 | 72 | 84 | 130 |
| CITsi_AFP1 | 25 | 76 | 1 | 31 | NA | NA | 32 | 72 | 76 | 137 |
| CITsi_AFP2 | 26 | 77 | 1 | 18 | NA | NA | 19 | 65 | 76 | 123 |
| CUCme_AFP1 | 27 | 78 | 1 | 8 | NA | NA | 9 | 56 | 69 | 115 |
| CUCsa_AFP3 | 28 | 79 | 1 | 8 | NA | NA | 9 | 56 | 69 | 115 |
| Gm_AFP1 | 29 | 80 | 1 | 27 | NA | NA | 28 | 69 | 73 | 137 |
| Gm_AFP2 | 30 | 81 | 1 | 27 | NA | NA | 28 | 69 | 82 | 129 |
| MALdo_AFP11 | 31 | 82 | 1 | 29 | NA | NA | 30 | 73 | 84 | 136 |
| Mt_AFP14 | 32 | 83 | 1 | 29 | NA | NA | 30 | 79 | 87 | 136 |
| Mt_AFP60 | 33 | 84 | 1 | 28 | NA | NA | 29 | 68 | 82 | 130 |
| Mt_AFP65 | 34 | 85 | 1 | 26 | NA | NA | 27 | 68 | 79 | 128 |
| Mt_AFP66 | 35 | 86 | 1 | 23 | NA | NA | 24 | 35 | 48 | 95 |
| Mt_AFP67 | 36 | 87 | 1 | 26 | NA | NA | 27 | 69 | 79 | 129 |
| Mt_AFP77 | 37 | 88 | 1 | 27 | NA | NA | 28 | 63 | 78 | 130 |
| Mt_AFP78 | 38 | 89 | 1 | 26 | NA | NA | 27 | 69 | 80 | 128 |
| PORol_AFP1 | 39 | 90 | 1 | 21 | NA | NA | 22 | 76 | 86 | 120 |
| PORol_AFP2 | 40 | 91 | NA | NA | NA | NA | 1 | 55 | 65 | 99 |
| PORol_AFP27 | 41 | 92 | 1 | 29 | NA | NA | 30 | 73 | 81 | 130 |
| PRUpe_AFP1 | 42 | 93 | 1 | 6 | NA | NA | 7 | 67 | 71 | 134 |
| RAPra_AFP23 | 43 | 94 | 1 | 32 | NA | NA | 33 | 76 | 89 | 137 |
| RAPra_AFP24 | 44 | 95 | 1 | 26 | NA | NA | 27 | 71 | 80 | 128 |
| RAPra_AFP25 | 45 | 96 | 1 | 9 | NA | NA | 10 | 55 | 67 | 115 |
| RAPra_AFP26 | 46 | 97 | 1 | 3 | NA | NA | 4 | 44 | 55 | 98 |
| RAPra_AFP27 | 47 | 98 | 1 | 26 | NA | NA | 27 | 67 | 78 | 121 |
| ROSbl_AFP11 | 48 | 99 | 1 | 26 | NA | NA | 11 | 68 | 72 | 120 |
| TRIre_AFP5 | 49 | 100 | 1 | 28 | NA | NA | 29 | 68 | 81 | 129 |
| Zm_AFP101 | 50 | 101 | 1 | 8 | 967 | 1002 | 9 | 40 | 95 | 140 |
| Zm_AFP100 | 51 | 102 | 1 | 54 | 421 | 474 | 55 | 104 | 133 | 178 |

| | D3 | | D4 | | L1 | | L2 | | L3 | |
|---|---|---|---|---|---|---|---|---|---|---|
| | D3 | D3 | D4 | D4 | Linker1 | Linker1 | Linker2 | Linker2 | Linker3 | Linker3 |
| Locus ID | Start | End | Start | End | Start | End | Start | End | Start | End |
| ARAly_AFP26 | | | | | 70 | 76 | | | | |
| At_AFP10 | | | | | 79 | 86 | | | | |
| At_AFP11 | | | | | 71 | 80 | | | | |
| At_AFP12 | | | | | 66 | 73 | | | | |
| At_AFP8 | | | | | 69 | 86 | | | | |
| At_AFP9 | | | | | 71 | 75 | | | | |
| BASsc_AFP21 | | | | | 55 | 64 | | | | |
| Bn_AFP35 | | | | | 77 | 86 | | | | |
| Bn_AFP36 | | | | | 51 | 62 | | | | |
| Bn_AFP47 | | | | | 69 | 79 | | | | |
| Bn_AFP74 | | | | | 71 | 79 | | | | |
| Bn_AFP75 | | | | | 61 | 71 | | | | |
| Bn_AFP76 | | | | | 71 | 79 | | | | |
| Bn_AFP79 | | | | | 65 | 74 | | | | |
| Bn_AFP80 | | | | | 65 | 74 | | | | |
| Br_AFP1 | | | | | 60 | 80 | | | | |
| Br_AFP2 | | | | | 67 | 73 | | | | |
| CARpa_AFP1 | | | | | 65 | 72 | | | | |
| CARpa_AFP2 | | | | | 70 | 77 | | | | |
| CARpa_AFP3 | | | | | 65 | 67 | | | | |
| CITcl_AFP10 | | | | | 67 | 69 | | | | |
| CITcl_AFP11 | | | | | 66 | 73 | | | | |
| CITcl_AFP2 | | | | | 76 | 82 | | | | |
| CITcl_AFP3 | | | | | 73 | 83 | | | | |
| CITsi_AFP1 | | | | | 73 | 75 | | | | |
| CITsi_AFP2 | | | | | 66 | 75 | | | | |
| CUCme_AFP1 | | | | | 57 | 68 | | | | |
| CUCsa_AFP3 | | | | | 57 | 68 | | | | |
| Gm_AFP1 | | | | | 70 | 72 | | | | |
| Gm_AFP2 | | | | | 70 | 81 | | | | |
| MALdo_AFP11 | | | | | 74 | 83 | | | | |
| Mt_AFP14 | | | | | 80 | 86 | | | | |
| Mt_AFP60 | | | | | 69 | 81 | | | | |
| Mt_AFP65 | | | | | 69 | 78 | | | | |
| Mt_AFP66 | | | | | 36 | 47 | | | | |
| Mt_AFP67 | | | | | 70 | 78 | | | | |
| Mt_AFP77 | | | | | 64 | 77 | | | | |
| Mt_AFP78 | | | | | 70 | 79 | | | | |
| PORol_AFP1 | | | | | 77 | 85 | | | | |
| PORol_AFP2 | | | | | 56 | 64 | | | | |
| PORol_AFP27 | | | | | 74 | 80 | | | | |
| PRUpe_AFP1 | | | | | 68 | 70 | | | | |
| RAPra_AFP23 | | | | | 77 | 88 | | | | |
| RAPra_AFP24 | | | | | 72 | 79 | | | | |
| RAPra_AFP25 | | | | | 56 | 66 | | | | |
| RAPra_AFP26 | | | | | 45 | 54 | | | | |
| RAPra_AFP27 | | | | | 68 | 77 | | | | |
| ROSbl_AFP11 | | | | | 69 | 71 | | | | |
| TRIre_AFP5 | | | | | 69 | 80 | | | | |
| Zm_AFP101 | | | | | 41 | 94 | | | | |
| Zm_AFP100 | 205 | 250 | 277 | 322 | 105 | 132 | 179 | 204 | 251 | 276 |

### Example 3. Identification of Linker Regions from Multi-Domain Defensins

Upon examination of the discovered multi-domain plant defensins, linker regions were identified between the defensin regions. The unique linker regions identified from the 2D and MD defensins are shown in Table 3. SEQ ID NOs: 103-152 represent nucleotide sequences encoding these identified defensin linker regions, and SEQ ID NOs: 153-202 represent the corresponding polypeptide sequences of these defensin linker regions. As can be seen in Table 3, there were 50 unique linker region DNA and protein sequences identified from these MD defensins since two of the linker regions for the 4D defensin (Zm_AFP100) are the same (SEQ ID NOs: 152 and 202). N-terminal TS sequences and C-terminal extension sequences (when present) for each of these identified MD defensins are further shown by sequence position annotations in Tables 1 and 2 above. The lengths of the TS sequences (if present) were observed to vary from a few amino acids to over 30 amino acids, and the C-terminal extension sequence was generally absent from the identified 2D defensins but was present in a 2D defensin (Zm_AFP101) and a 4D defensin (Zm_AFP100) at a variable length of about 35-50 amino acids. Most of the linkers were found to be proline-rich, while others were enriched with glycine or charged amino acids. Many of the linkers were approximately 10-20 amino acids in length, but a small subset of linkers had either very short linkers of only a few amino acids or longer linker sequence lengths of 20-65 amino acids.

TABLE 32
D and MD Defensin Linker Regions.

| Locus ID | Linker ID | DNA SEQ ID NO | Protein SEQ ID NO |
|---|---|---|---|
| ARAly_AFP26 | ARAly26L10 | 103 | 153 |
| At_AFP10 | At10L18 | 104 | 154 |
| At_AFP11 | At11L19 | 105 | 155 |
| At_AFP12 | At12L20 | 106 | 156 |
| At_AFP8 | At8L21 | 107 | 157 |
| At_AFP9 | At9L22 | 108 | 158 |
| BASsc_AFP21 | BASsc21L23 | 109 | 159 |
| Bn_AFP35 | Bn35L11 | 110 | 160 |
| Bn_AFP36 | Bn36L1 | 111 | 161 |
| Bn_AFP47 | Bn47L2 | 112 | 162 |
| Bn_AFP74 | Bn74L3 | 113 | 163 |
| Bn_AFP75 | Bn75L13 | 114 | 164 |
| Bn_AFP76 | Bn76L3 | 113 | 163 |
| Bn_AFP79 | Bn79L7 | 115 | 165 |
| Bn_AFP80 | Bn80L7 | 115 | 165 |
| Br_AFP1 | Br1L24 | 116 | 166 |
| Br_AFP2 | Br2L25 | 117 | 167 |
| CARpa_AFP1 | CARpa1L26 | 118 | 168 |
| CARpa_AFP2 | CARpa2L27 | 119 | 169 |
| CARpa_AFP3 | CARpa3L28 | 120 | 170 |
| CITcl_AFP10 | CITcl10L29 | 121 | 171 |
| CITcl_AFP11 | CITcl11L30 | 122 | 172 |
| CITcl_AFP2 | CITcl2L17 | 123 | 173 |
| CITcl_AFP3 | CITcl3L14 | 124 | 174 |
| CITsi_AFP1 | CITsi1L31 | 125 | 175 |
| CITsi_AFP2 | CITsi2L32 | 126 | 176 |
| CUCme_AFP1 | CUCme1L33 | 127 | 177 |
| CUCsa_AFP3 | CUCsa3L34 | 128 | 178 |
| Gm_AFP1 | Gm1L35 | 129 | 179 |
| Gm_AFP2 | Gm2L36 | 130 | 180 |
| MALdo_AFP11 | MALdo11L12 | 131 | 181 |
| Mt_AFP14 | Mt14L6 | 132 | 182 |
| Mt_AFP60 | Mt60L4 | 133 | 183 |
| Mt_AFP65 | Mt65L8 | 134 | 184 |
| Mt_AFP66 | Mt66L15 | 135 | 185 |
| Mt_AFP67 | Mt67L16 | 136 | 186 |
| Mt_AFP77 | Mt77L5 | 137 | 187 |
| Mt_AFP78 | Mt78L9 | 138 | 188 |
| PORol_AFP1 | PORol1L37 | 139 | 189 |
| PORol_AFP2 | PORol2L38 | 140 | 190 |
| PORol_AFP27 | PORol27L39 | 141 | 191 |
| PRUpe_AFP1 | PRUpe1L40 | 142 | 192 |
| RAPra_AFP23 | RAPra23L41 | 143 | 193 |
| RAPra_AFP24 | RAPra24L42 | 144 | 194 |
| RAPra_AFP25 | RAPra25L43 | 145 | 195 |
| RAPra_AFP26 | RAPra26L44 | 146 | 196 |
| RAPra_AFP27 | RAPra27L45 | 147 | 197 |
| ROSbl_AFP11 | ROSbl11L46 | 148 | 198 |
| TRIre_AFP5 | TRIre5L47 | 149 | 199 |
| Zm_AFP101 | Zm101L49 | 150 | 200 |
| Zm_AFP100 L1 | Zm100L48 | 151 | 201 |
| Zm_AFP100 L2 | Zm100L50 | 152 | 202 |
| Zm_AFP100 L3 | Zm100L50 | 152 | 202 |

### Example 4. Design of Synthetic Multi-Domain Defensins

In this example, synthetic homodimeric 2D defensins were designed and constructed to comprise two copies of a cognate single domain (1D) defensin, for example MtDef4 (nucleotide SEQ ID NO: 1029; polypeptide SEQ ID NO: 1128) or Coix22 (nucleotide SEQ ID NO: 990; polypeptide SEQ ID NO: 1089), connected by various linker regions derived from native 2D or MD defensins described herein. Synthetic multi-domain defensins comprising Coix22 defensin regions and having one of the nucleic acid sequences of SEQ ID NOs: 203-219 (encoding the protein sequences of SEQ ID NOs: 329-345, respectively) linked or connected by 17 different linker regions (identified in Table 4) were assessed in corn protoplasts. Synthetic multi-domain defensins comprising MtDef4 defensin regions and having nucleic acid sequences of SEQ ID NOs: 220-236 (encoding the protein sequences of SEQ ID NOs: 346-362, respectively) with the same linker regions as identified in Table 4 were also assessed in corn and soy protoplasts. Further reference is made to Tables 3 and 5 for the sequence identifiers corresponding to these 17 linker regions.

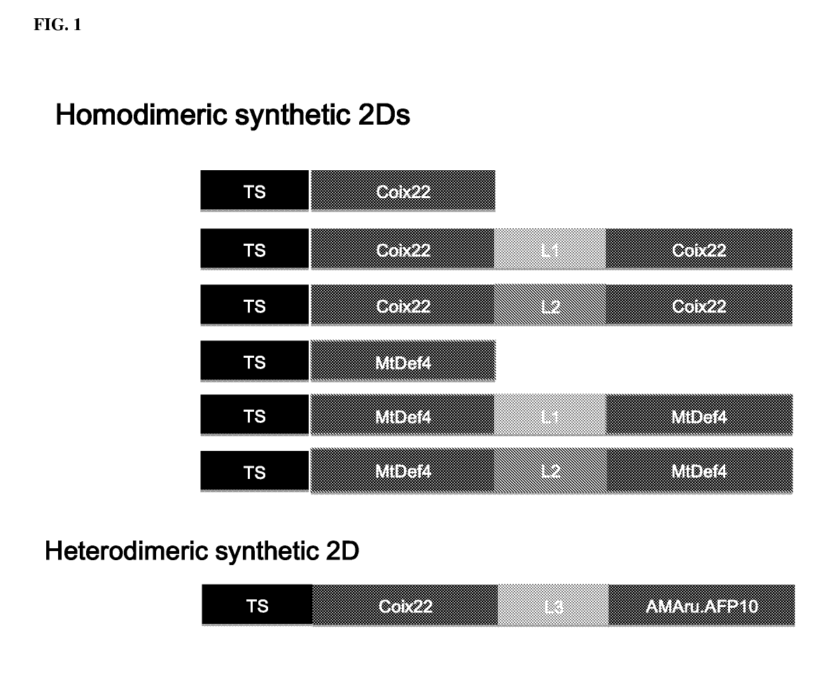

Also described in this example is the design of a synthetic heterodimeric defensin as set forth in nucleotide SEQ ID NO: 1155 and polypeptide SEQ ID NO: 1156 using Coix 22, linker Mt.AFP65 (nucleotide SEQ ID NO: 134; polypeptide SEQ ID NO: 184) and AMAru.AFP10 (nucleotide SEQ ID NO: 963; polypeptide SEQ ID NO: 1062). Stop codons were removed from the N-terminal defensin region of these synthetic multi-domain 2D defensins. The defensin N-terminal transit signal (TS) domain was maintained at the N-terminus of the N-terminal defensin region, but removed from the N-terminus of the C-terminal defensin region. Diagrammatic examples of these synthetic defensins are shown in FIG. 1 (L1, L2 and L3 refer to linkers derived from different 2D defensins identified herein).

TABLE 4
Homodimeric synthetic 2D using MtDef4 or Coix22 defensins

| Nucle- | | | | | | | | | |
|---|---|---|---|---|---|---|---|---|---|
| otide | Protein | | | Protein | Protein | Protein | Protein | Protein | Protein |
| SEQ | SEQ | | | TS | TS | MP | MP | Linker | Linker |
| ID NO | ID NO | Template ID | Linker ID | Start | End | Start | End | Start | End |
| 203 | 329 | Cl22FL_ARAly26L10_Cl22D1 | ARAly26L10 | 1 | 31 | 32 | 80 | 81 | 87 |
| 204 | 330 | Cl22FL_Bn35L11_Cl22D1 | Bn35L11 | 1 | 31 | 32 | 80 | 81 | 90 |
| 205 | 331 | Cl22FL_Bn36L1_Cl22D1 | Bn36L1 | 1 | 31 | 32 | 80 | 81 | 92 |
| 206 | 332 | Cl22FL_Bn47L2_Cl22D1 | Bn47L2 | 1 | 31 | 32 | 80 | 81 | 91 |
| 207 | 333 | Cl22FL_Bn74L3_Cl22D1 | Bn74L3 | 1 | 31 | 32 | 80 | 81 | 89 |
| 208 | 334 | Cl22FL_Bn75L1_Cl22D1 | Bn75L13 | 1 | 31 | 32 | 80 | 81 | 91 |
| 209 | 335 | Cl22FL_Bn79L7_Cl22D1 | Bn79L7 | 1 | 31 | 32 | 80 | 81 | 90 |
| 210 | 336 | Cl22FL_CITcl2L17_Cl22D1 | CITcl2L17 | 1 | 31 | 32 | 80 | 81 | 87 |
| 211 | 337 | Cl22FL_CITcl3L14_Cl22D1 | CITcl3L14 | 1 | 31 | 32 | 80 | 81 | 91 |
| 212 | 338 | Cl22FL_MALdo11L12_Cl22D1 | MALdo11L12 | 1 | 31 | 32 | 80 | 81 | 90 |
| 213 | 339 | Cl22FL_Mt14L6_Cl22D1 | Mt14L6 | 1 | 31 | 32 | 80 | 81 | 87 |
| 214 | 340 | Cl22FL_Mt60L4_Cl22D1 | Mt60L4 | 1 | 31 | 32 | 80 | 81 | 93 |
| 215 | 341 | Cl22FL_Mt65L8_Cl22D1 | Mt65L8 | 1 | 31 | 32 | 80 | 81 | 90 |
| 216 | 342 | Cl22FL_Mt66L15_Cl22D1 | Mt66L15 | 1 | 31 | 32 | 80 | 81 | 92 |
| 217 | 343 | Cl22FL_Mt67L16_Cl22D1 | Mt67L16 | 1 | 31 | 32 | 80 | 81 | 89 |
| 218 | 344 | Cl22FL_Mt77L5_Cl22D1 | Mt77L5 | 1 | 31 | 32 | 80 | 81 | 94 |
| 219 | 345 | Cl22FL_Mt78L9_Cl22D1 | Mt78L9 | 1 | 31 | 32 | 80 | 81 | 90 |
| 220 | 346 | MtDef4FL_ARAly26L10_MtDef4D1 | ARAly26L10 | 1 | 29 | 30 | 76 | 77 | 83 |
| 222 | 348 | MtDef4FL_Bn36L1_MtDef4D1 | Bn36L1 | 1 | 29 | 30 | 76 | 77 | 88 |
| 223 | 349 | MtDef4FL_Bn47L2_MtDef4D1 | Bn47L2 | 1 | 29 | 30 | 76 | 77 | 87 |
| 224 | 350 | MtDef4FL_Bn74L3_MtDef4D1 | Bn74L3 | 1 | 29 | 30 | 76 | 77 | 85 |
| 225 | 351 | MtDef4FL_Bn75L13_MtDef4D1 | Bn75L13 | 1 | 29 | 30 | 76 | 77 | 87 |
| 226 | 352 | MtDef4FL_Bn79L7_MtDef4D1 | Bn79L7 | 1 | 29 | 30 | 76 | 77 | 86 |
| 227 | 353 | MtDef4FL_CITcl2L17_MtDef4D1 | CITcl2L17 | 1 | 29 | 30 | 76 | 77 | 83 |
| 228 | 354 | MtDef4FL_CITcl3L14_MtDef4D1 | CITcl3L14 | 1 | 29 | 30 | 76 | 77 | 87 |
| 229 | 355 | MtDef4FL_MALdo11L12_MtDef4D1 | MALdo11L12 | 1 | 29 | 30 | 76 | 77 | 86 |
| 230 | 356 | MtDef4FL_Mt14L6_MtDef4D1 | Mt14L6 | 1 | 29 | 30 | 76 | 77 | 83 |
| 231 | 357 | MtDef4FL_Mt60L4_MtDef4D1 | Mt60L4 | 1 | 29 | 30 | 76 | 77 | 89 |
| 232 | 358 | MtDef4FL_Mt65L8_MtDef4D1 | Mt65L8 | 1 | 29 | 30 | 76 | 77 | 86 |
| 233 | 359 | MtDef4FL_Mt66L15_MtDef4D1 | Mt66L15 | 1 | 29 | 30 | 76 | 77 | 88 |
| 234 | 360 | MtDef4FL_Mt67L16_MtDef4D1 | Mt67L16 | 1 | 29 | 30 | 76 | 77 | 85 |
| 235 | 361 | MtDef4FL_Mt77L5_MtDef4D1 | Mt77L5 | 1 | 29 | 30 | 76 | 77 | 90 |
| 236 | 362 | MtDef4FL_Mt78L9_MtDef4D1 | Mt78L9 | 1 | 29 | 30 | 76 | 77 | 86 |

| Nucle- | | | | | | | | | | |
|---|---|---|---|---|---|---|---|---|---|---|
| otide | Protein | Protein | DNA | DNA | DNA | DNA | DNA | DNA | DNA | DNA |
| SEQ | MP2 | MP2 | TS | TS | MP | MP | Linker | Linker | MP2 | MP2 |
| ID NO | Start | End | Start | End | Start | End | Start | End | Start | End |
| 203 | 88 | 136 | 1 | 93 | 94 | 240 | 241 | 261 | 262 | 408 |
| 204 | 91 | 139 | 1 | 93 | 94 | 240 | 241 | 270 | 271 | 417 |
| 205 | 93 | 141 | 1 | 93 | 94 | 240 | 241 | 276 | 277 | 423 |
| 206 | 92 | 140 | 1 | 93 | 94 | 240 | 241 | 273 | 274 | 420 |
| 207 | 90 | 138 | 1 | 93 | 94 | 240 | 241 | 267 | 268 | 414 |
| 208 | 92 | 140 | 1 | 93 | 94 | 240 | 241 | 273 | 274 | 420 |
| 209 | 91 | 139 | 1 | 93 | 94 | 240 | 241 | 270 | 271 | 417 |
| 210 | 88 | 136 | 1 | 93 | 94 | 240 | 241 | 261 | 262 | 408 |
| 211 | 92 | 140 | 1 | 93 | 94 | 240 | 241 | 273 | 274 | 420 |
| 212 | 91 | 139 | 1 | 93 | 94 | 240 | 241 | 270 | 271 | 417 |
| 213 | 88 | 136 | 1 | 93 | 94 | 240 | 241 | 261 | 262 | 408 |
| 214 | 94 | 142 | 1 | 93 | 94 | 240 | 241 | 279 | 280 | 426 |
| 215 | 91 | 139 | 1 | 93 | 94 | 240 | 241 | 270 | 271 | 417 |
| 216 | 93 | 141 | 1 | 93 | 94 | 240 | 241 | 276 | 277 | 423 |
| 217 | 90 | 138 | 1 | 93 | 94 | 240 | 241 | 267 | 268 | 414 |
| 218 | 95 | 143 | 1 | 93 | 94 | 240 | 241 | 282 | 283 | 429 |
| 219 | 91 | 139 | 1 | 93 | 94 | 240 | 241 | 270 | 271 | 417 |
| 220 | 84 | 130 | 1 | 87 | 88 | 228 | 229 | 249 | 250 | 390 |
| 222 | 89 | 135 | 1 | 87 | 88 | 228 | 229 | 264 | 265 | 405 |
| 223 | 88 | 134 | 1 | 87 | 88 | 228 | 229 | 261 | 262 | 402 |
| 224 | 86 | 132 | 1 | 87 | 88 | 228 | 229 | 255 | 256 | 396 |
| 225 | 88 | 134 | 1 | 87 | 88 | 228 | 229 | 261 | 262 | 402 |
| 226 | 87 | 133 | 1 | 87 | 88 | 228 | 229 | 258 | 259 | 399 |
| 227 | 84 | 130 | 1 | 87 | 88 | 228 | 229 | 249 | 250 | 390 |
| 228 | 88 | 134 | 1 | 87 | 88 | 228 | 229 | 261 | 262 | 402 |
| 229 | 87 | 133 | 1 | 87 | 88 | 228 | 229 | 258 | 259 | 399 |
| 230 | 84 | 130 | 1 | 87 | 88 | 228 | 229 | 249 | 250 | 390 |
| 231 | 90 | 136 | 1 | 87 | 88 | 228 | 229 | 267 | 268 | 408 |
| 232 | 87 | 133 | 1 | 87 | 88 | 228 | 229 | 258 | 259 | 399 |
| 233 | 89 | 135 | 1 | 87 | 88 | 228 | 229 | 264 | 265 | 405 |
| 234 | 86 | 132 | 1 | 87 | 88 | 228 | 229 | 255 | 256 | 396 |
| 235 | 91 | 137 | 1 | 87 | 88 | 228 | 229 | 270 | 271 | 411 |
| 236 | 87 | 133 | 1 | 87 | 88 | 228 | 229 | 258 | 259 | 399 |

### Example 5. Protein Accumulation of 1D Versus 2D Defensins in Corn and Soy Plant Cells

### A. Protein Accumulation in Corn Protoplasts

Select synthetic homodimeric 2D defensins described in Example 4 were synthesized and delivered in a pUC57 cloning vector. Polymerase chain reaction (PCR) amplification and restriction digestion were used to amplify sequences encoding the synthetic 2D defensins from the pUC57 vectors, which were then ligated into a protoplast expression vector comprising a 35S promoter linked to the synthetic 2D defensin coding sequences. Protoplasts isolated from corn leaf mesophyll tissues were transformed with the protoplast expression plasmid vectors expressing native 1D Coix22 or synthetic 2D defensins comprising Coix22 defensin domains/regions connected by a linker region. For these synthetic 2D constructs, the homodimeric Coix22 defensin regions were heterologous with respect to the linker region. Following transformation, the protoplasts were lysed and the total proteins were subjected to standard Western immunoblotting using rabbit polyclonal antibodies raised against the native 1D Coix22. A goat anti-rabbit antibody conjugated to horseradish peroxidase (HRP) was used as secondary antibody and the blots were visualized with the Super Signal West Femto kit (Thermo Fisher Scientific, Waltham, Mass.). Following Western blotting, the membranes were subjected to Coomassie staining to confirm equal sample loading. Dilutions of a purified full length CI_AFP22 were western blotted and the resulting bands were subject to densitometry imaging (Imager Lab 5.1) and used to create a standard curve. The standard curve was then used as a basis for comparison to quantify the 1D and 2D CI_AFP22 proteins that were expressed in corn protoplasts.

Protein accumulation of homodimeric Coix22 domains in combination with various heterologous linker sequences (see Table 4) were compared with protein accumulation for Coix22 1D defensin components. In these experiments, many of the synthetic 2D defensin proteins appear to accumulate to greater levels than their 1D defensin counterparts. Although one of the synthetic 2D defensins tested, homodimeric Coix22 2D linked by Mt77L5 (nucleic acid SEQ ID NO: 218; protein SEQ ID NO: 344), was cleaved in both soy and corn protoplasts, no cleavage product was observed for the remainder of the synthetic 2D Coix22 defensins tested. Accordingly, it is proposed that linker regions may be leveraged to enhance accumulation of 2D and MD defensins in plants to enable more effective control of plant pathogens and/or pests.

### B. Protein Accumulation in Transgenic Soybean Plants

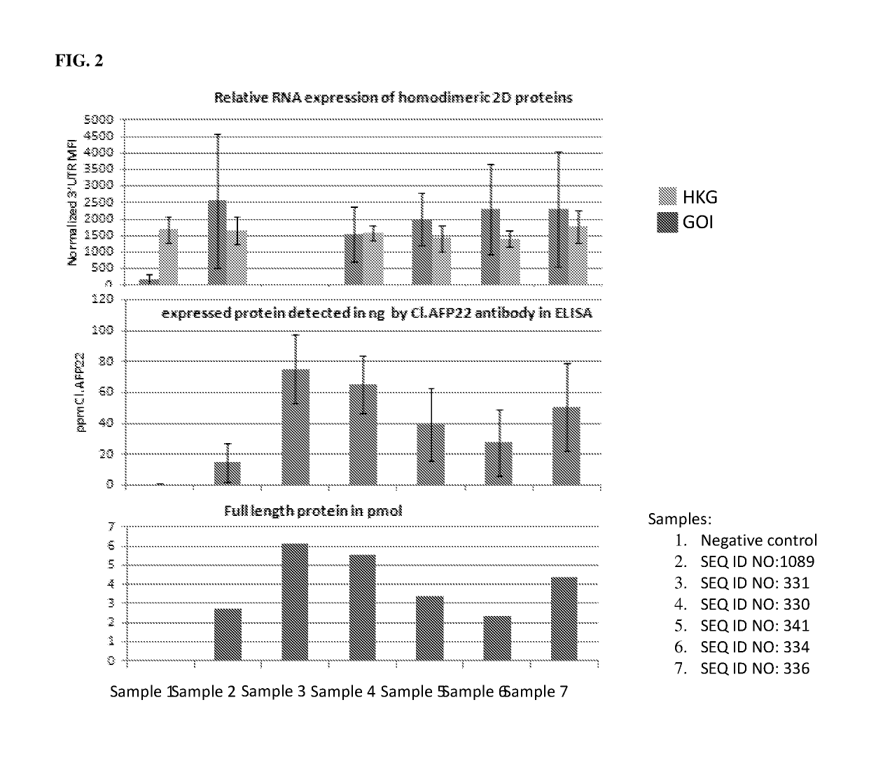

Transgenic soybean plants expressing Coix22 and Six Coix22 homodimeric 2D defensins with different heterologous linkers were established, and samples were collected at V2 and lyophilized prior to RNA and protein quantification. Protein quantification was performed using an indirect ELISA with a rabbit IgG primary antibody raised against the protein of interest and an anti-rabbit goat secondary antibody. Two concentrations of standard protein were used to calculate protein detected which is then normalized by total protein extracted. The QuantiGene technology measures RNA directly via a nucleic acid hybridization platform in which target RNAs are captured through cooperative binding of multiple oligonucleotide probes that are conjugated to magnetic microbeads. Cooperative binding of the multiple oligonucleotide probes with specificity for the target sequence results in exceptionally high assay specificity. Detection of this oligonucleotide complex occurs through amplification of a branched DNA amplifier and fluorescent signal, which is counted digitally by high-throughput flow cytometry sorting of the microbead. The results of RNA and Protein levels are shown in FIG. 2. While the variance in RNA is not significant between the 1D coix22 and the homodimeric Coix22 2D defensins, three of the five 2D proteins expressed at higher levels than the single domain, even after normalizing for the difference in protein size (2.25-fold increase for the 2D defensin using linker Bn36L1, 2-fold for the 2D defensin using linker Bn35L1, and 1.6-fold for the 2D defensin using linker CITcl2L17). These results demonstrate that multiple domains may help in protein accumulation through increased stability in transgenic soybean plants.

### Example 6. Methods for Assessing Activity of Multi-Domain Defensins

In this example, synthetic 2D defensins and their 1D defensin counterparts were tested for their ability to inhibit growth of several fungal species. The defensins were expressed in Pichia yeast cells and purified for use in fungal plate assays as described below.

### Defensin Production Using Pichia Cells

Constructs designed for Pichia and containing a Pichia signal peptide were synthesized by Bio Basic Inc. For clarity, this “Pichia signal peptide” used for these experiments is different than the N-terminal TS sequence of defensin proteins and may be present in addition to the TS sequence. The Pichia signal peptide is used to enable secretion of the synthesized protein from the Pichia cells to facilitate collection and protein purification. Approximately 5 μg of DNA was linearized, purified, and transformed into electrocompetent PichiaPink™ cells (Thermo Scientific) by electroporation. After recovery, the cells were plated onto Pichia Adenine Dropout (PAD) selection plates and incubated at 29° C. for 3 days. One colony from each transformation was cultured for 3 days for outgrowth. The cell pellets were then collected and induced with media containing methanol. The cells were cultured and supplemented with methanol daily for 3 additional days. The cell pellets were collected and the media used for the purification. Methods for purification of the Pichia-expressed defensin proteins depended on their expected isoelectric point (pI).

Purification of Defensins Having pI>7 Using Cation Exchange.

Media was diluted at a ratio of 8:1 with 50 mM sodium acetate at pH 5.0 and passed over SP sepharose resin (GE Healthcare #17-0729-01) using a vacuum manifold. The resin was washed with 50 mM sodium acetate at pH 5.0 and eluted in twice the resin volume with 50 mM sodium acetate at pH 5.0 containing 1M NaCl.

Purification of Defensins Having pI<7 Using Anion Exchange.

Media was diluted at a ratio of 8:1 with 50 mM CHES at pH 9.0 and passed over Q sepharose resin (GE Healthcare #17-0510-01) using a vacuum manifold. The resin was washed with 50 mM CHES at pH 9.0 and eluted in twice the resin volume with 50 mM CHES at pH 9.0 containing 1M NaCl.

Buffer Exchange and Concentration.

The purified protein was dialyzed overnight into 8 mM Tris HCl at pH 8 using Pura-A-Lyzer Maxi 3500 Dialysis tubes (Sigma #PURX35050). The resulting solution was concentrated by drying in the Pura-A-Lyzer tubes until a final volume of 400-450 μl was achieved.

### Fungal Growth Conditions

Fungi were first grown according to the conditions in Table 5 for 3-4 weeks or less depending on fungal species as indicated.

TABLE 6
Fungal Growth Conditions.

| | | Lighting and Temperature | |
|---|---|---|---|
| Fungus | Media | Conditions | Time before use |
| Fusarium | 0.25x Potato | 12 hour diurnal cycle at 19° C. | 21-28 days |
| graminearum | Dextrose Agar | in the dark and 23° C. with | |
| (Gibberella stalk rot) | | 100 μmol light | |
| Fusarium | 0.25x Potato | 12 hour diurnal cycle at 19° C. | 21-28 days |
| verticilloides | Dextrose Agar | in the dark and 23° C. with | |
| (Fusarium stalk rot) | | 100 μmol light | |
| Colletotrichum | 1x oatmeal | 12 hour diurnal cycle at 19° C. | 21-28 days |
| graminicola | agar | in the dark and 23° C. with | |
| (Anthracnose stalk | | 100 μmol light | |
| rot) | | | |
| Stenocarpella maydis | 1X PDA + | 12 hour diurnal cycle at 26° C. | 21-28 days |
| (Diplodia ear rot) | Cefotaxime | with 100 μmol of light | |
| | | during the daytime period | |
| Phakopsora | Culture in | 14 hour diurnal light cycle | At least 12 days after |
| pachyrhizi (Soy Asian | planta | with 19° C. in the dark and | plant inoculation |
| rust) | | 22° C. with 500 μmol light | |
| | | during the daytime period, | |
| | | 75% relative humidity | |

### Plate Functional Assay for Fungal Growth Inhibition

Following expression and purification of the 1D and 2D defensin proteins and growth of the fungal species in culture, the ability of the individual defensins to inhibit fungal growth was tested in plate assays. On the day of the growth test assay, fungi were collected in test media, filtered, and resuspended to the appropriate titer as shown in Table 7.

TABLE 7
Conditions for Fungal Growth Inhibition Assay.

| | Test | Filter | | |
|---|---|---|---|---|
| Fungus | media | condition | Test media | Titer |
| Fusarium | 0.05% | 100 μm | 0.5x POTATO | 4e⁴ |
| graminearum | Triton | Steriflip | DEXTROSE | spores/ml |
| (Gibberella stalk rot) | X100 | filter | BROTH | |
| Fusarium verticilloides | 0.05% | 100 μm | 0.5x POTATO | 6.5e⁴ |
| (Fusarium stalk rot) | Triton | Steriflip | DEXTROSE | spores/ml |
| | X100 | filter | BROTH | |
| Colletotrichum | 0.05% | 100 μm | 2x POTATO | 1e⁵ |
| graminicola | Triton | Steriflip | DEXTROSE | spores/ml |
| (Anthracnose stalk rot) | X100 | filter | BROTH | |
| Stenocarpella maydis | 0.05% | 100 μm | 2x POTATO | 6.5e⁴ |
| (Diplodia ear rot) | Triton | Steriflip | DEXTROSE | spores/ml |
| | X100 | filter | BROTH | |
| Phakopsora pachyrhizi | 0.05% | 20 μm and 60 | 0.5x POTATO | 5e⁴ |
| (Soy Asian rust) | Triton | μm Steriflip | DEXTROSE | spores/ml |
| | X100 | filter | BROTH | |

20 μl of each fungal solution according to Table 7 was added to 20 μl of the purified defensin protein to be tested in 20 mM Tris HCL, pH 8.0, in 96 well view plates (Perkin Elmer). Plates were sealed with parafilm and incubated in a clear humidity box for a predetermined period of time, temperature and conditions according to Table 8, before being imaged for fungal growth in the presence of the defensin.

TABLE 8
Conditions and Times for Incubation of Fungi with Defensins.

| | Lighting and | |
|---|---|---|
| Fungus | Temperature Conditions | Time |
| Fusarium | 12 hour diurnal cycle at 23° C. | 24 hours |
| graminearum | with 100 μmol of light during | |
| (Gibberella stalk rot) | the daytime period | |
| Fusarium | 12 hour diurnal cycle at 23° C. | 24 hours |
| verticilloides | with 100 μmol of light during | |
| (Fusarium stalk rot) | the daytime period | |
| Colletotrichum | 12 hour diurnal cycle at 26° C. | 24 hours |
| graminicola | with 100 μmol of light during | |
| (Anthracnose stalk rot) | the daytime period | |
| Stenocarpella maydis | 12 hour diurnal cycle at 26° C. | 24 hours |
| (Diplodia ear rot) | with 100 μmol of light during | |
| | the daytime period | |
| Phakopsora | 12 hour diurnal cycle at 23° C. | 16 hours |
| pachyrhizi | with 100 μmol of light during | |
| (Soy Asian rust) | the daytime period | |

### Imaging Fungal Growth

The plates were visually imaged using the Perkin Elmer Operetta High Content Imager, using brightfield settings and a 10× objective lens. Depending on the particular fungal species being tested, the stored images were scored according to the ratings or criteria provided in Table 9. Two replicates and up to 4 fields per replicates were averaged for each of the visual inhibition scores.

TABLE 9
Scoring of Inhibition of Fungal Growth.

| Fungus | Scoring |
|---|---|
| Fusarium graminearum | 0-Germinated, hyphal elongation and no activity |
| (Gibberella stalk rot) | 1-Germinated and hyphal elongation of ≥250 μm |
| | 2-Germinated and hyphal elongation of 100-250 μm |
| | 3-germinated and hyphal elongation of 50-100 μm |
| | 4-no germination >80% of spores; hyperbranching “starburst” phenotype |
| | with hyphal elongation <50 μm |
| Fusarium verticilloides | 0-Germinated, hyphal elongation and no activity |
| (Fusarium stalk rot) | 1-Germinated and hyphal elongation of ≥400 μm |
| | 2-Germinated and hyphal elongation of 200-400 μm |
| | 3-germinated and hyphal elongation of 100-200 μm |
| | 4-no germination >50% of spores; hyperbranching “starburst” phenotype |
| | with hyphal elongation <100 μm |
| Colletotrichum | 0-Germinated and hyphal elongation ≥ 400 μm |
| graminicola | 1-Germinated and hyphal elongation of 200-400 μm |
| (Anthracnose stalk rot) | 2-Germinated and hyphal elongation of 125-200 μm |
| | 3-germinated and hyphal elongation of <125 μm |
| | 4-no germination |
| Stenocarpella maydis | 0-Germinated and hyphal elongation ≥ 400 μm |
| (Diplodia ear rot) | 1-Germinated and hyphal elongation of 200-400 μm |
| | 2-Germinated and hyphal elongation of 125-200 μm |
| | 3-germinated and hyphal elongation of <125 μm |
| | 4-activity with no germination >25% of spores; |
| | hyphal elongation < 25 μm |
| Phakopsora pachyrhizi | 0-0% Inhabitation ≥ 330 μm |
| (Soy Asian rust) | 1-25% Inhabitation 330-250 um |
| | 2-20-75% Inhabitation 100-250 μm |
| | 3-75-100% Inhabitation 0-100 μm |

### Example 7. Fungal Inhibition Activity of Multi-Domain Defensins

A. 2D Defensins Inhibit Fungal Growth in Plate Assay

Eighteen native 2D defensins of the invention were tested in the above in vitro plate assay and compared with their 1D defensin counterparts to determine whether the 2D defensins are active against the fungal pathogens listed in Table 9. These 19 2D defensins are identified in Table 10, and their sequences are provided above in Tables 1 and 2 (their linker sequences are also provided in Table 3; and corresponding protein sequences may be determined from the nucleotide sequences listed). The anti-fungal activities of the 2D defensins were also compared to their cognate or component 1D defensins to determine if their potency and spectrum of activity were enhanced relative to their 1D components. The 1D or 2D defensins tested were expressed in Pichia and purified as described above. The fungal inhibition scores for each defensin were calculated as an average of the reps performed.

TABLE 10
Fungal Inhibition Activity of 2D Defensins.

| NUC | | | | | Colletot- | | Fusarium | | | |
|---|---|---|---|---|---|---|---|---|---|---|
| SEQ | protein | | | | richum | Fusarium | verticil- | Phakopsora | Stenocarpella | |
| ID NO | name | locus ID | Domain | Reps | graminicola | graminearum | lioides | pachyrhizi | maydis | >20 ppm |
| 1 | PHT001688 | ARAly_AFP26 | D1D2 | 2 | 0 | 1 | 0 | 0 | 1 | not |
| | | | | | | | | | | known |
| 8 | PHT002159 | Bn_AFP35 | D1D2 | 2 | 0 | 0 | 0 | 0 | 0 | not |
| | | | | | | | | | | known |
| 9 | PHT002230 | Bn_AFP36 | D1D2 | 2 | 0 | 0 | 0 | | | not |
| | | | | | | | | | | known |
| 10 | PHT002187 | Bn_AFP47 | D1D2 | 2 | 0 | 0 | 0 | 0 | 0.5 | not |
| | | | | | | | | | | known |
| 11 | PHT002172 | Bn_AFP74 | D1D2 | 2 | 0 | 0 | 0 | 0 | 0 | not |
| | | | | | | | | | | known |
| 12 | PHT002235 | Bn_AFP75 | D1D2 | 2 | 0 | 0 | 0 | | 0 | not |
| | | | | | | | | | | known |
| 13 | PHT002236 | Bn_AFP76 | D1D2 | 2 | 0 | 0 | 0 | 0 | 0 | not |
| | | | | | | | | | | known |
| 14 | PHT002182 | Bn_AFP79 | D1D2 | 2 | 0 | 0 | 0 | 0 | 0.5 | not |
| | | | | | | | | | | known |
| 15 | PHT002219 | Bn_AFP80 | D1D2 | 2 | 0 | 0 | 0 | 0 | 0 | not |
| | | | | | | | | | | known |
| 24 | PHT002366 | CITcl_AFP3 | D1D2 | 2 | 0 | 0 | 0 | 0.5 | 1.5 | not |
| | | | | | | | | | | known |
| 31 | PHT002131 | MAFdo_AFP11 | D1D2 | 2 | 0 | 1.5 | 0.5 | 1.5 | 1 | yes |
| 32 | PHT002270 | Mt_AFP14 | D1D2 | 2 | 0 | 4 | 2.5 | | 2 | yes |
| 33 | PHT002295 | Mt_AFP60 | D1D2 | 2 | 0 | 0 | 0 | 0 | 0 | not |
| | | | | | | | | | | known |
| 34 | PHT002294 | Mt_AFP65 | D1D2 | 2 | 0 | 0.5 | 0 | 0 | 0 | not |
| | | | | | | | | | | known |
| 35 | PHT002293 | Mt_AFP66 | D1D2 | 2 | 0 | 0 | 0 | 0 | 0 | not |
| | | | | | | | | | | known |
| 37 | PHT002296 | Mt_AFP77 | D1D2 | 2 | 0 | 1 | 0 | 0 | 0.5 | yes |
| 38 | PHT002297 | Mt_AFP78 | D1D2 | 2 | 0 | 0 | 0 | 0 | 1 | not |
| | | | | | | | | | | known |
| — | Empty Vector | negative control | N/A | 4 | 0 | 0.25 | 0 | 0 | 1.5 | no |
| 1153 | MEDsa.AFPm1 | positive control | N/A | 4 | 2.75 | 3.7 | 2.3 | 1.25 | 2.7 | yes |

As shown in Table 10, some of these native 2D defensins were able to inhibit growth of fungal pathogens tested in this plate functional assay. Protein concentrations (ppm) of some of the defensin preparations used in the fungal growth assay were measured and compared. In this experiment, at least one of the different 2D defensins was found to have activity against each of the fungal species tested. In cases where the protein concentration was measured and determined to be greater than 20 ppm, inhibitory activity against one or more pathogens was observed for that defensin protein.

B. Transgenic Corn Plants Expressing MALdo_AFP11 have Enhanced Disease Resistance

Transgenic corn plants expressing MALdo_AFP11 (nucleotide SEQ ID NO: 31 and polypeptide SEQ ID NO: 82) were generated and grown in the greenhouse. At the VT growth stage, two nodes were infected by wounding the stalk and injecting a suspension of Colletotrichum graminicola inoculum into the wound. Corn plants were evaluated ˜2-3 weeks after inoculation. Stalks were harvested, leaves were removed and the stalks were split longitudinally. Disease severity was reported as the percent necrosis of the cut surface and is compared to the percent necrosis of non-transformed plants. Five transgenic corn events out of 9 transgenic corn events tested were shown to have significant disease reduction at p-value <0.05 as compared to controls.

### Example 8. Fungal Inhibition by Synthetic Multi-Domain Defensins

Synthetic 2D defensins comprising two identical defensin regions connected or bridged together by a linker region were compared with 1D defensins. As shown in Example 5 above, many of these longer 2D defensins exhibited increased accumulation relative to their 1D counterparts when expressed in the corn and soy protoplast system, perhaps due to their increased size. The synthetic 2D defensins that accumulated to higher levels in protoplasts also tended to have enhanced or altered activity against fungal pathogens in the plate assays.

Coix22 (PHT000006; nucleotide SEQ ID NO: 990; polypeptide SEQ ID NO: 1089) and MtDef4 (PHT000025; nucleotide SEQ ID NO: 1029; polypeptide SEQ ID NO: 1128) are 1D defensins shown to be active against fungal pathogens. These Coix22 and MtDef4 domains were fused with heterologous defensin linker regions to create novel synthetic 2D defensins similar to those expressed in Examples 4 and 5 above but modified for expression in Pichia. Seventeen unique defensin linkers from 2D defensins identified from different plant species were tested for their ability to create active 2D defensins. Each synthetic defensin was expressed from a Pichia expression vector, and proteins were purified and tested in vitro for activity as described above. The 1D Coix22 and MtDef4 defensins were used as controls. The fungal inhibition activities or scores for these synthetic 2D defensins are shown in Tables 11, 12, and 13. Tables 12 and 13 provide these fungal inhibition activities or scores standardized to 1 μM or 0.5 μM protein concentration. Standardization of these fungal inhibition scores allows for a more direct comparison of the intrinsic anti-fungal activities of these 2D and 1D defensins, apart from the effects of their variable protein accumulation levels.

TABLE 11
Fungal Inhibition Activity of Synthetic 2D defensins.

| Defensin | | | final | estimate | | | | | |
|---|---|---|---|---|---|---|---|---|---|
| PRT SEQ | Protein | | assay | of Pichia | C. | F. | F. | P. | S. |
| ID NO | name | linker | cone (μM) | processing | graminicola | graminearum | verticilloides | pachyrhizi | maydis |
| — | Empty Vector | (Empty) | 5 | (Empty) | 0 | 1.5 | 0.5 | 0 | 1 |
| 1154 | MEDsa.AFPm1 | (Empty) | 3.72 | (Empty) | 4 | 3 | 3 | 3 | 3 |
| 1089 | PHT000006 | none | 5 | N/A | 3 | 1 | 0 | 2 | 2.5 |
| 365 | PHT002411 | PPTPPSPFrRP | 5 | not cleaved | 3 | 2.5 | 2.5 | 2 | 1.5 |
| 366 | PHT002412 | GPPSPTPPHHK | 5 | cleaved | 3.5 | 1 | 2 | 1 | 2.5 |
| 373 | PHT002416 | APKKVEP | 3.96 | not cleaved | 3 | 2 | 1 | 2 | 0.5 |
| 369 | PHT002417 | NNESASPASK | 1.75 | not cleaved | 2.5 | 2 | 1 | 1 | 1 |
| 375 | PHT002418 | GGKAGKKAPK | 5 | not cleaved | 2.5 | 4 | 3.5 | 2 | 1 |
| 363 | PHT002420 | ASIKPAK | 2.86 | cleaved | 2 | 1 | 2 | 0.5 | 2 |
| 364 | PHT002529 | ATPPTPTPPK | 5 | not cleaved | 3 | 3 | 2.5 | 2 | 0.5 |
| 372 | PHT002530 | GPPSPPPYSK | 5 | cleaved | 2 | 1.5 | 0.5 | 2 | 3 |
| 368 | PHT002531 | EPPSLTSTPLN | 5 | not cleaved | 2 | 2.5 | 2 | 2 | 0.5 |
| 377 | PHT002534 | GGKPGKKAP | 1.1 | not cleaved | 1 | 1.5 | 1 | 0 | 2.5 |
| 370 | PHT002535 | AGRGDKK | 5 | not cleaved | 2 | 1 | 1 | 0.5 | 2.5 |
| — | Tris | (Empty) | (Empty) | (Empty) | 0.9 | 0.7 | 0 | 0.3 | 0.4 |

TABLE 12
Fungal Inhibition Activity of Synthetic 2D Defensins Standardized to a Protein Concentration of 1 μM.

| Defensin | | | final | estimate | | | | | |
|---|---|---|---|---|---|---|---|---|---|
| PRT SEQ | Protein | | assay | of Pichia | C. | F. | F. | P. | S. |
| ID NO | name | linker | cone (μM) | processing | graminicola | graminearum | verticilloides | pachyrhizi | maydis |
| — | Empty Vector | (Empty) | 1 | (Empty) | 1.5 | 1 | 0 | 0 | 1 |
| 1154 | MEDsa.AEPm1 | (Empty) | 1 | (Empty) | 2.5 | 3 | 2.5 | 0.5 | 3 |
| 1089 | PHT000006 | none | 1 | N/A | 0.5 | 0 | 1 | 0.5 | 2 |
| 365 | PHT002411 | PPTPPSPPTRP | 1 | not cleaved | 1.5 | 0 | 1 | 1 | 3 |
| 366 | PHT002412 | GPPSPTPPHHK | 1 | cleaved | 1.5 | 0 | 1 | 1 | 2.5 |
| 373 | PHT002416 | APKKVEP | 1 | not cleaved | 1 | 1 | 2 | 0.5 | 3 |
| 369 | PHT002417 | NNESASPASK | 1 | not cleaved | 1.5 | 1 | 1 | 0 | 1.5 |
| 375 | PHT002418 | GGKAGKKAPK | 1 | not cleaved | 2 | 1 | 1.5 | 0 | 2 |
| 363 | PHT002420 | ASIKPAK | 1 | cleaved | 2 | 1 | 1.5 | 0.5 | 1.5 |
| 364 | PHT002529 | ATPFrrrppK | 1 | not cleaved | 2.5 | 0 | 1 | 1 | 2 |
| 372 | PHT002530 | GPPSPPPYSK | 1 | cleaved | 1.5 | 1 | 0.5 | 0.5 | 1.5 |
| 368 | PHT002531 | EPPSLTSTPLN | 1 | not cleaved | 1.5 | 0 | 1 | 1 | 1 |
| 377 | PHT002534 | GGKPGKKAP | 1 | not cleaved | 0 | 1 | 0.5 | 0 | 0 |
| 370 | PHT002535 | AGRGDKK | 1 | not cleaved | 1 | 0 | 0 | 0 | 1.5 |

TABLE 13
Fungal Inhibition Activity of Synthetic 2D Defensins Standardized to a Protein Concentration of 0.5 μM.

| Defensin | | | final | estimate | | | | | |
|---|---|---|---|---|---|---|---|---|---|
| PRT SEQ | Protein | | assay | of Pichia | C. | F. | F. | P. | S. |
| ID NO | name | linker | cone (μM) | processing | graminicola | graminearum | verticilloides | pachyrhizi | maydis |
| — | Empty Vector | (Empty) | 0.5 | (Empty) | 1 | 0 | 0 | 0 | 1.5 |
| 1154 | MEDsa.AFPm1 | (Empty) | 0.5 | (Empty) | 1 | 3 | 1.5 | 1 | 2.5 |
| 1089 | PHT000006 | none | 0.5 | N/A | 2.5 | 0.5 | 1 | 1 | 1 |
| 365 | PHT002411 | PPTPPSPPTRP | 0.5 | not cleaved | 2.5 | 0 | 1 | 1 | 2 |
| 366 | PHT002412 | GPPSPTPPHHK | 0.5 | cleaved | 2.5 | 0.5 | 1 | 0.5 | 2 |
| 373 | PHT002416 | APKKVEP | 0.5 | not cleaved | 0.5 | 0 | 1.5 | 1 | 1 |
| 369 | PHT002417 | NNESASPASK | 0.5 | not cleaved | 2.5 | 0 | 1 | 1.5 | 2.5 |
| 375 | PHT002418 | GGKAGKKAPK | 0.5 | not cleaved | 1 | 0.5 | 1 | 1 | 2 |
| 363 | PHT002420 | ASFKPAK | 0.5 | cleaved | 1 | 1 | 1.5 | 0.5 | 2.5 |
| 364 | PHT002529 | ATPPTPTPPK | 0.5 | not cleaved | 2 | 1 | 0.5 | 1.5 | 2.5 |
| 372 | PHT002530 | GPPSPPPYSK | 0.5 | cleaved | 0.5 | 0 | 1 | 1 | 2 |
| 368 | PHT002531 | EPPSLTSTPLN | 0.5 | not cleaved | 0 | 0 | 1 | 0 | 2 |
| 377 | PHT002534 | GGKPGKKAP | 0.5 | not cleaved | 0.5 | 1 | 1 | 0 | 1 |
| 370 | PHT002535 | AGRGDKK | 0.5 | not cleaved | 1 | 1 | 0 | 0 | 2.5 |

### Example 9. Fungal Inhibition by Synthetic Multi-Domain Defensins

As demonstrated in Example 8, defensin regions can be connected by a linker region sequence to create a functionally active synthetic or chimeric 2D defensin and tested for anti-fungal activity. Seventeen linker regions from different 2D defensins were tested in combination with five native 2D defensin regions by replacing the native linker region with one of the 16 heterologous linkers (one of the 17 linkers was the native linker for that 2D defensin). The five native 2D defensins were chosen from a set of 17 2D defensins based on the cysteines in the 2 defensin regions. Two defensins, Mt_AFP14 and Bn_AFP79, represented the most common 8:8 class of 2D defensins. Bn_AFP79 and Mt_AFP65 represented the 8:10 class, and Mt_AFP60 represented the 7:9 class of defensins. See Tables 1 and 2 providing sequence identifiers for these native 2D proteins. For 2D defensins, this x:y nomenclature refers to the relative number of cysteines in the two defensin regions. For example, the 8:10 class refers to a 2D defensin having 8 cysteines in the first defensin region and 10 cysteines in the second defensin region. A total of 80 constructs (16 heterologous linker regions in 5 native 2D defensins) and three controls were tested for fungal inhibition activity in the plate assay. Data available for 57 of these constructs is shown in Table 14 below. Each of the five native 2D defensins were also tested (i.e., without the linker swap). The results are shown in Table 14. Proteins were expressed in Pichia and purified as described above.

TABLE 14
Fungal Inhibition Activity of Synthetic Chimeric 2D Defensins.

| NUC | PRT | | Colletot- | | Fusarium | | | |
|---|---|---|---|---|---|---|---|---|
| SEQ | SEQ | | richum | Fusarium | verticil- | Stenocarpella | Phakopsora | protein |
| ID NO | ID NO | template_id | graminicola | graminearum | lioides | maydis | pachyrhizi | expression |
| 14 | 65 | BRANA.G1156000011.1 | 0 | 0 | 0 | 0 | 0 | N/A |
| 10 | 61 | BRANA.G181000052.1 | 0 | 0 | 0 | 0.5 | 0 | N/A |
| 32 | 83 | contig_21756_1.1 | 0 | 2.5 | 0 | 0 | 0 | N/A |
| 33 | 84 | Medtr2g037250.1 | 0 | 1.5 | 0 | 0 | 0 | N/A |
| 34 | 85 | Medtr2g037190.1 | 0 | 1 | 0 | 0 | 0 | N/A |
| 271 | 397 | Bn47D1_Bn35L11_Bn47D2 | | 0 | 0 | | 0 | band not |
| | | | | | | | | visible |
| 272 | 398 | Bn47D1_Bn36L1_Bn47D2 | 0 | 0 | 0 | 0 | 0 | band not |
| | | | | | | | | visible |
| 273 | 399 | Bn47D1_MALdo11L12_Bn47D2 | 0 | 0 | 0 | 0 | 0 | band not |
| | | | | | | | | visible |
| 274 | 400 | Bn47D1_Bn74L3_Bn47D2 | 0 | 0 | 0 | 0 | 0 | band not |
| | | | | | | | | visible |
| 275 | 401 | Bn47D1_Bn75L13_Bn47D2 | 0 | 0 | 0 | 2 | 0 | band not |
| | | | | | | | | visible |
| 276 | 402 | Bn47D1_Mt60L4_Bn47D2 | 0 | 0 | 0 | 0 | 0 | band not |
| | | | | | | | | visible |
| 277 | 403 | Bn47D1_Mt77L5_Bn47D2 | | 0 | 0 | 0 | 0 | band not |
| | | | | | | | | visible |
| 278 | 404 | Bn47D1_CITcl3L14_Bn47D2 | | 0 | 0 | | 0 | + |
| 279 | 405 | Bn47D1_Mt14L6_Bn47D2 | 0 | 0 | 0 | 0 | 0 | band not |
| | | | | | | | | visible |
| 280 | 406 | Bn47D1_Mt66L15_Bn47D2 | 0 | 0 | 0 | 0 | 0 | + |
| 281 | 407 | Bn47D1_Mt65L8_Bn47D2 | 0 | 0 | 0 | 0 | 0 | band not |
| | | | | | | | | visible |
| 282 | 408 | Bn47D1_Mt67L16_Bn47D2 | 0 | 0 | 0 | 0 | 0 | + |
| 283 | 409 | Bn47D1_Mt78L9_Bn47D2 | 0 | 0 | 0 | 0 | 0 | band not |
| | | | | | | | | visible |
| 284 | 410 | Bn47D1_CITcl2L17_Bn47D2 | 0 | 0 | 0 | 0 | 0 | band not |
| | | | | | | | | visible |
| 285 | 411 | Mt60D1_Bn36L1_Mt60D2 | 0 | 0 | 0 | 0 | 0 | band not |
| | | | | | | | | visible |
| 286 | 412 | Mt60D1_Bn47L2_Mt60D2 | 0 | 0 | 0 | 0 | 0 | + |
| 287 | 413 | Mt60D1_MALdo11L12_Mt60D2 | 0 | 0 | 0 | 0 | 0 | + |
| 288 | 414 | Mt60D1_Bn75L13_Mt60D2 | 0 | 0 | 0 | 3 | 0 | band not |
| | | | | | | | | visible |
| 289 | 415 | Mt60D1_Mt77L5_Mt60D2 | 0 | 0 | 0 | 0 | 0 | ++ |
| 290 | 416 | Mt60D14_CITcl3L14_Mt60D2 | 0 | 2 | 0 | 0 | 0 | ++ |
| 291 | 417 | Mt60D1_Mt14L6_Mt60D2 | 0 | 0 | 0 | 0 | 0 | band not |
| | | | | | | | | visible |
| 292 | 418 | Mt60D1_Bn79L7_Mt60D2 | 0 | 0 | 0 | 0 | 0 | band not |
| | | | | | | | | visible |
| 293 | 419 | Mt60D1_Mt66L15_Mt60D2 | 0 | 0 | 0 | 0 | 0 | ++ |
| 294 | 420 | Mt60D1_Mt65L8_Mt60D2 | 0 | 0.5 | 0 | 0 | 0 | ++ |
| 295 | 421 | Mt60D1_Mt78L9_Mt60D2 | 0 | 0 | 0 | 0 | 0 | + |
| 296 | 422 | Mt60D1_CITcl2L17_Mt60D2 | 0 | 0 | 0 | 0 | 0 | ++ |
| 297 | 423 | Mt14D1_Bn47L2_Mt14D2 | 0 | 2.5 | 1 | 0 | 0 | band not |
| | | | | | | | | visible |
| 298 | 424 | Mt14D1 _MALdo11L12_Mt14D2 | 0 | 1.5 | 1 | 0 | 0 | band not |
| | | | | | | | | visible |
| 299 | 425 | Mt14D1_Bn74L3_Mt14D2 | 0 | 4 | 1.5 | 1.5 | 0 | band not |
| | | | | | | | | visible |
| 300 | 426 | Mt14D1_Bn75L13_Mt14D2 | 0 | 2 | 0 | 0 | 0 | band not |
| | | | | | | | | visible |
| 301 | 427 | Mt14D1_Mt60L4_Mt14D2 | 0 | 3 | 1 | 1 | 0 | band not |
| | | | | | | | | visible |
| 302 | 428 | Mt14D1_Mt77L5_Mt14D2 | 0 | 1 | 0 | 0 | 0 | band not |
| | | | | | | | | visible |
| 303 | 429 | Mt14D1_CITcl3L14_Mt14D2 | 0 | 2.5 | 1.5 | 0 | 0 | ++ |
| 304 | 430 | Mt14D1_Mt78L9_Mt14D2 | 0 | 3.5 | 1.5 | 0 | 0 | band not |
| | | | | | | | | visible |
| 305 | 431 | Mt14D1_CITcl2L17_Mt14D2 | 0 | 0 | 0 | 0 | 0 | band not |
| | | | | | | | | visible |
| 306 | 432 | Bn79D1_Bn35L11_Bn79D2 | 0 | 0 | 0 | 0 | 0 | band not |
| | | | | | | | | visible |
| 307 | 433 | Bn79D1_Bn47L2_Bn79D2 | 0 | 0 | 0 | 0 | 0 | + |
| 308 | 434 | Bn79D1_Bn74L3_Bn79D2 | 0 | 0 | 0 | 0 | 0 | + |
| 309 | 435 | Bn79D1_Bn75L13_Bn79D2 | 0 | 0 | 0 | 0 | 0 | band not |
| | | | | | | | | visible |
| 310 | 436 | Bn79D1_Mt60L4_Bn79D2 | 0 | 0 | 0 | 0 | 0 | band not |
| | | | | | | | | visible |
| 311 | 437 | Bn79D1_Mt77L5_Bn79D2 | 0 | 0 | 0 | 0 | 0 | + |
| 312 | 438 | Bn79D1_Mt14L6_Bn79D2 | 0 | 0 | 0 | 0 | 0 | + |
| 313 | 439 | Bn79D1_Mt66L15_Bn79D2 | 0 | 0 | 0 | 0 | 0 | ++ |
| 314 | 440 | Bn79D1_Mt65L8_Bn79D2 | 0 | 0 | 0 | 0 | 0 | + |
| 315 | 441 | Bn79D1_Mt78L9_Bn79D2 | 0 | 0 | 0 | 0 | 0 | + |
| 316 | 442 | Bn79D1_CITcl2L17_Bn79D2 | 0 | 0 | 0 | 2.5 | 0 | + |
| 317 | 443 | Bn79D1_ARAly26L10_Bn79D2 | 0 | 0 | 0 | 0 | 0 | failed |
| | | | | | | | | transformation |
| 318 | 444 | Mt65D1_Bn35L11_Mt65D2 | 0 | 2 | 0 | 0 | 0 | + |
| 319 | 445 | Mt65D1_Bn36L1_Mt65D2 | 0 | 0 | 0 | 0 | 0 | band not |
| | | | | | | | | visible |
| 320 | 446 | Mt65D1_MALdo11L12_Mt65D2 | 0 | 2 | 1 | 0 | 0 | band not |
| | | | | | | | | visible |
| 321 | 447 | Mt65D1_Bn75L13_Mt65D2 | 0 | 0 | 0 | 0 | 0 | band not |
| | | | | | | | | visible |
| 322 | 448 | Mt65D1_Mt77L5_Mt65D2 | 0 | 0 | 0 | 0 | 0 | band not |
| | | | | | | | | visible |
| 323 | 449 | Mt65D1_CITcl3L14_Mt65D2 | 0 | 0.5 | 0 | 0 | 0 | band not |
| | | | | | | | | visible |
| 324 | 450 | Mt65D1_Bn79L7_Mt65D2 | 0 | 0 | 0 | | 0 | band not |
| | | | | | | | | visible |
| 325 | 451 | Mt65D1_Mt66L15_Mt65D2 | 0 | 0 | 0 | 0 | 0 | + |
| 326 | 452 | Mt65D1_Mt78L9_Mt65D2 | 0 | 0 | 0 | 0 | 0 | band not |
| | | | | | | | | visible |
| 327 | 453 | Mt65D1_CITcl2L17_Mt65D2 | 0 | 0 | 0 | 0 | 0 | band not |
| | | | | | | | | visible |
| 328 | 454 | Mt65D1_ARAly26L10_Mt65D2 | 0 | 0 | 0 | 0 | 0 | band not |
| | | | | | | | | visible |
| | | MEDsa.AFPm1 | 3 | 3 | 2.25 | 2.6 | 2 | N/A |
| | | Empty Vector | 0 | 0 | 0 | 0.5 | 0 | N/A |
| | | 20 mM Tris-HCl pH 8.0 | 0 | 0 | 0 | 0 | 0 | N/A |

This data demonstrates that linkers have a broad capacity for linking two defensin regions together to create synthetic 2D defensin that are active against one or more fungal pathogens. Eight of the seventeen linkers tested (Bn35L11, Bn47L2, Bn74L3, Bn75L13, CITcl2L17, CITcl3L14, MALdo11L12, and Mt65L8) showed promise in working across different defensin backgrounds. Many of the synthetic multi-domain or 2D defensins had higher levels of protein accumulation in plant protoplasts with the potential for new or altered spectrums of activity against different fungal pathogens.

### Example 10. Disease Reduction by Synthetic Heterodimeric 2D Defensins in Transgenic Soybean Plants

Transgenic soybean plants expressing heterodimeric 2D defensin Cl.AFP22/linker Mt.AFP65/AMAru.AFP10 (nucleotide SEQ ID NO: 1155 and polypeptide SEQ ID NO: 1156) were established and grown in soil in growth chambers and inoculated with a pathogen suspension containing Phakopsora pachyrhizi (pathogen for Asian soy rust, ASR), at the V2 stage. The plants were scored 12-14 days after inoculation for percent leaf infection. The score was compared to that of plants transformed with an empty vector. The transgenic soybean plants expressing the heterodimeric 2D defensin showed significant disease reduction as compared to the control plants.

TABLE 15
Transgenic soybean plants expressing heterodimeric 2D defensin.

| | | Numbers | | |
|---|---|---|---|---|
| | NUC SEQ ID NO/ | of Sample | Mean of percent leaf | Std |
| Sample ID | PRT SEQ ID NO | tested | infection * | Error |
| Empty Vector Control | / | 33 | 41.8182 | 1.7029 |
| Cl.AFP22/linker | 1155/1156 | 30 | 31.3333 | 1.7860 |
| Mt.AFP65/AMAru.AFP10 | | | | |

* Means are statistically different at p < 0.05

### Example 11. 1D Defensin Capable of Inhibiting Fungal Growth

Several native 1D defensins were also identified that may be useful either alone or as defensin regions/domains when designing synthetic 2D or other MD defensins. Table 16 provides a list of native 1D defensins with nucleotide and protein sequence identifiers and annotated to identify the boundaries between different sequence regions or domains. The ninety-nine (99) defensins listed in Table 16 were selected based on their activity in the fungal plate assay described above in Example 6. Of the 413 1D defensin proteins tested, the 99 1D defensins listed in Table 16 had an average fungal growth inhibition score of 2 or greater over at least two repeated experiments against at least one of the five fungi listed in the Tables above.

As similarly described above, the polynucleotide sequence positions defining the boundaries of the polynucleotide sequences encoding the N-terminal TS sequence (TS_Start, TS_End), the defensin sequence portion (Def_Start, Def_End), and the C-terminal extension sequence (CT_Start, Ct_End; if present) are identified for each of the 1D defensins listed. Based on the information provided in Table 16, one can discern the corresponding protein sequence boundaries of the N-terminal TS sequence, defensin sequence portion, and C-terminal extension sequence (if present) of a 1D defensin encoded by the polynucleotide sequences in Table 15 by dividing the nucleotide positions by three (an amino acid corresponding to one codon of three nucleotides). As an exemplary illustration, the 1D defensin protein “ARAly_AFP15” has an amino acid length of 83 amino acids with its TS sequence corresponding to amino acids 1-19, its defensin sequence portion corresponding to amino acids 20-75, and its C-terminal extension sequence corresponding to amino acids 76-83 of SEQ ID NO: 1054.

TABLE 16
Identification of 1D defensins.

| | | | Targeting Signal (TS) Sequence | | | | | | | |
|---|---|---|---|---|---|---|---|---|---|---|
| | DNA | PRT | DNA | PRT | | | | | | |
| | SEQ | SEQ | SEQ | SEQ | | | | | | |
| Locus_ID | ID NO | ID NO | ID NO | ID NO | TS_Start | TS_End | Def_Start | Def_End | CT_Start | CT_End |
| ARAly_AFP15 | 955 | 1054 | 713 | 859 | 1 | 57 | 58 | 225 | 226 | 249 |
| Zm_AFP82 | 956 | 1055 | 714 | 860 | 1 | 81 | 82 | 237 | 238 | 321 |
| IPOtr_AFP2 | 957 | 1056 | 715 | 861 | 1 | 96 | 97 | 237 | No CT | No CT |
| ABUth_AFP1 | 958 | 1057 | 716 | 862 | 1 | 81 | 82 | 222 | No CT | No CT |
| ABUth_AFP6 | 959 | 1058 | 717 | 863 | 1 | 24 | 25 | 180 | No CT | No CT |
| AMBar_AFP1 | 960 | 1059 | 718 | 864 | 1 | 93 | 94 | 234 | No CT | No CT |
| AMBar_AFP7 | 961 | 1060 | 719 | 865 | 1 | 117 | 118 | 249 | No CT | No CT |
| AMAru_AFP16 | 962 | 1061 | 720 | 866 | 1 | 90 | 91 | 240 | No CT | No CT |
| AMAru_AFP10 | 963 | 1062 | 721 | 867 | 1 | 81 | 82 | 240 | No CT | No CT |
| AMBtr_AFP6 | 964 | 1063 | 722 | 868 | 1 | 213 | 214 | 360 | No CT | No CT |
| AMBtr_AFP7 | 965 | 1064 | 723 | 869 | 1 | 93 | 94 | 234 | No CT | No CT |
| BRAdi_AFP13 | 966 | 1065 | 724 | 870 | 1 | 96 | 97 | 240 | No CT | No CT |
| Bn_AFP43 | 967 | 1066 | 725 | 871 | 1 | 78 | 79 | 240 | 241 | 255 |
| Bn_AFP1 | 968 | 1067 | 726 | 872 | 1 | 75 | 76 | 231 | 232 | 240 |
| Bn_AFP51 | 969 | 1068 | 727 | 873 | 1 | 90 | 91 | 267 | 268 | 282 |
| Bn_AFP98 | 970 | 1069 | 728 | 874 | 1 | 87 | 88 | 252 | 253 | 264 |
| Bn_AFP52 | 971 | 1070 | 729 | 875 | 1 | 231 | 232 | 402 | 403 | 417 |
| Bn_AFP103 | 972 | 1071 | 730 | 876 | 1 | 81 | 82 | 228 | 229 | 273 |
| Sb_AFP1 | 973 | 1072 | 731 | 877 | 1 | 78 | 79 | 231 | 232 | 384 |
| CANro_AFP2 | 974 | 1073 | 732 | 878 | 1 | 78 | 79 | 219 | No CT | No CT |
| CANro_AFP1 | 975 | 1074 | 733 | 879 | 1 | 93 | 94 | 234 | No CT | No CT |
| CANro_AFP13 | 976 | 1075 | 734 | 880 | 1 | 84 | 85 | 213 | 214 | 273 |
| CHEal_AFP7 | 977 | 1076 | 735 | 881 | 1 | 108 | 109 | 249 | No CT | No CT |
| CHEal_AFP1 | 978 | 1077 | 736 | 882 | 1 | 78 | 79 | 222 | No CT | No CT |
| CONca_AFP1 | 979 | 1078 | 737 | 883 | 1 | 96 | 97 | 237 | No CT | No CT |
| STEme_AFP1 | 980 | 1079 | 738 | 884 | 1 | 96 | 97 | 231 | No CT | No CT |
| ERAte_AFP11 | 981 | 1080 | 739 | 885 | 1 | 87 | 88 | 234 | No CT | No CT |
| Mt_AFP52 | 982 | 1081 | 740 | 886 | 1 | 9 | 10 | 171 | No CT | No CT |
| ERAte_AFP14 | 983 | 1082 | 741 | 887 | 1 | 90 | 91 | 276 | No CT | No CT |
| ERAte_AFP23 | 984 | 1083 | 742 | 888 | 1 | 90 | 91 | 255 | 256 | 303 |
| ERAte_AFP26 | 985 | 1084 | 743 | 889 | 1 | 87 | 88 | 258 | No CT | No CT |
| ERAte_AFP29 | 986 | 1085 | 744 | 890 | 1 | 87 | 88 | 249 | 250 | 291 |
| ERAte_AFP30 | 987 | 1086 | 745 | 891 | 1 | 66 | 67 | 207 | 208 | 303 |
| ERAte_AFP36 | 988 | 1087 | 746 | 892 | 1 | 72 | 73 | 216 | 217 | 324 |
| ERAte_AFP37 | 989 | 1088 | 747 | 893 | 1 | 99 | 100 | 258 | 259 | 303 |
| Cl_AFP22 | 990 | 1089 | 748 | 894 | 1 | 93 | 94 | 240 | No CT | No CT |
| Cl_AFP24 | 991 | 1090 | 749 | 895 | 1 | 69 | 70 | 210 | 211 | 312 |
| ERAte_AFP56 | 992 | 1091 | 750 | 896 | 1 | 69 | 70 | 213 | 214 | 333 |
| ERAte_AFP57 | 993 | 1092 | 751 | 897 | 1 | 87 | 88 | 249 | No CT | No CT |
| Cl_AFP39 | 994 | 1093 | 752 | 898 | 1 | 12 | 13 | 186 | 187 | 228 |
| ERAte_AFP58 | 995 | 1094 | 753 | 899 | 1 | 60 | 61 | 219 | 220 | 354 |
| Cl_AFP44 | 996 | 1095 | 754 | 900 | 1 | 96 | 97 | 240 | No CT | No CT |
| ERAte_AFP65 | 997 | 1096 | 755 | 901 | 1 | 81 | 82 | 249 | No CT | No CT |
| STEme_AFP2 | 998 | 1097 | 756 | 902 | 1 | 30 | 31 | 162 | No CT | No CT |
| STEme_AFP6 | 999 | 1098 | 757 | 903 | 1 | 138 | 139 | 282 | No CT | No CT |
| ERAte_AFP87 | 1000 | 1099 | 758 | 904 | 1 | 87 | 88 | 234 | No CT | No CT |
| ERAte_AFP92 | 1001 | 1100 | 759 | 905 | 1 | 144 | 145 | 351 | No CT | No CT |
| STEme_AFP4 | 1002 | 1101 | 760 | 906 | 1 | 96 | 97 | 246 | No CT | No CT |
| ERAte_AFP94 | 1003 | 1102 | 761 | 907 | 1 | 90 | 91 | 276 | No CT | No CT |
| ERAte_AFP95 | 1004 | 1103 | 762 | 908 | 1 | 81 | 82 | 246 | 247 | 312 |
| ARAly_AFP1 | 1005 | 1104 | 763 | 909 | 1 | 87 | 88 | 231 | 232 | 246 |
| At_AFP1 | 1006 | 1105 | 764 | 910 | 1 | 90 | 91 | 231 | No CT | No CT |
| Ps_AFP1 | 1007 | 1106 | — | — | — | — | 1 | 138 | No CT | No CT |
| PINsy_AFP1 | 1008 | 1107 | 765 | 911 | 1 | 99 | 100 | 246 | 247 | 252 |
| Ta_AFP1 | 1009 | 1108 | — | — | — | — | 1 | 141 | No CT | No CT |
| Ph_AFP2 | 1010 | 1109 | 766 | 912 | 1 | 75 | 76 | 222 | 223 | 306 |
| DIGsa_AFP1 | 1011 | 1110 | 767 | 913 | 1 | 192 | 193 | 348 | 349 | 399 |
| SACra_AFP18 | 1012 | 1111 | 768 | 914 | 1 | 75 | 76 | 195 | 196 | 282 |
| SACra_AFP20 | 1013 | 1112 | 769 | 915 | 1 | 15 | 16 | 171 | 172 | 207 |
| SACra_AFP23 | 1014 | 1113 | 770 | 916 | 1 | 81 | 82 | 228 | 229 | 324 |
| SACra_AFP27 | 1015 | 1114 | 771 | 917 | 1 | 78 | 79 | 231 | 232 | 330 |
| EUPma_AFP6 | 1016 | 1115 | 772 | 918 | 1 | 87 | 88 | 237 | No CT | No CT |
| EUPma_AFP7 | 1017 | 1116 | 773 | 919 | 1 | 87 | 88 | 237 | No CT | No CT |
| EUPma_AFP2 | 1018 | 1117 | 774 | 920 | 1 | 81 | 82 | 222 | No CT | No CT |
| FOEvu_AFP4 | 1019 | 1118 | 775 | 921 | 1 | 102 | 103 | 273 | No CT | No CT |
| BASsc_AFP18 | 1020 | 1119 | 776 | 922 | 1 | 54 | 55 | 207 | 208 | 261 |
| BASsc_AFP20 | 1021 | 1120 | 777 | 923 | 1 | 90 | 91 | 246 | No CT | No CT |
| BASsc_AFP3 | 1022 | 1121 | 778 | 924 | 1 | 81 | 82 | 225 | No CT | No CT |
| BASsc_AFP25 | 1023 | 1122 | 779 | 925 | 1 | 36 | 37 | 351 | No CT | No CT |
| BASsc_AFP11 | 1024 | 1123 | 780 | 926 | 1 | 24 | 25 | 174 | No CT | No CT |
| Os_AFP12 | 1025 | 1124 | 781 | 927 | 1 | 69 | 70 | 213 | 214 | 321 |
| Os_AFP9 | 1026 | 1125 | 782 | 928 | 1 | 72 | 73 | 213 | 214 | 258 |
| Mt_AFP36 | 1027 | 1126 | 783 | 929 | 1 | 87 | 88 | 213 | 214 | 222 |
| Ta_AFP4 | 1028 | 1127 | — | — | — | — | 1 | 375 | No CT | No CT |
| Mt_Def4 | 1029 | 1128 | 784 | 930 | 1 | 87 | 88 | 228 | No CT | No CT |
| PANha_AFP7 | 1030 | 1129 | 785 | 931 | 1 | 96 | 97 | 237 | No CT | No CT |
| PANvi_AFP55 | 1031 | 1130 | 786 | 932 | 1 | 75 | 76 | 303 | No CT | No CT |
| PANvi_AFP17 | 1032 | 1131 | 787 | 933 | 1 | 69 | 70 | 216 | 217 | 369 |
| PLAma_AFP7 | 1033 | 1132 | 788 | 934 | 1 | 15 | 16 | 156 | 157 | 330 |
| PORol_AFP12 | 1034 | 1133 | 789 | 935 | 1 | 81 | 82 | 225 | No CT | No CT |
| PORol_AFP15 | 1035 | 1134 | 790 | 936 | 1 | 87 | 88 | 231 | No CT | No CT |
| PORol_AFP4 | 1036 | 1135 | 791 | 937 | 1 | 90 | 91 | 231 | No CT | No CT |
| PORol_AFP6 | 1037 | 1136 | 792 | 938 | 1 | 24 | 25 | 165 | No CT | No CT |
| RAPra_AFP6 | 1038 | 1137 | 793 | 939 | 1 | 81 | 82 | 219 | No CT | No CT |
| RAPra_AFP8 | 1039 | 1138 | 794 | 940 | 1 | 87 | 88 | 240 | No CT | No CT |
| RAPra_AFP9 | 1040 | 1139 | 795 | 941 | 1 | 87 | 88 | 240 | No CT | No CT |
| RAPra_AFP11 | 1041 | 1140 | 796 | 942 | 1 | 87 | 88 | 237 | No CT | No CT |
| RAPra_AFP20 | 1042 | 1141 | 797 | 943 | 1 | 87 | 88 | 237 | No CT | No CT |
| RAPra_AFP12 | 1043 | 1142 | 798 | 944 | 1 | 87 | 88 | 240 | No CT | No CT |
| ROSbl_AFP13 | 1044 | 1143 | 799 | 945 | 1 | 30 | 31 | 198 | No CT | No CT |
| ROSbl_AFP7 | 1045 | 1144 | 800 | 946 | 1 | 81 | 82 | 225 | No CT | No CT |
| ROSbl_AFP8 | 1046 | 1145 | 801 | 947 | 1 | 81 | 82 | 222 | No CT | No CT |
| Sb_AFP6 | 1047 | 1146 | 802 | 948 | 1 | 96 | 97 | 237 | No CT | No CT |
| SPIol_AFP13 | 1048 | 1147 | 803 | 949 | 1 | 93 | 94 | 249 | No CT | No CT |
| SPIol_AFP15 | 1049 | 1148 | 804 | 950 | 1 | 84 | 85 | 231 | No CT | No CT |
| TARof_AFP4 | 1050 | 1149 | 805 | 951 | 1 | 84 | 85 | 231 | 232 | 414 |
| TARof_AFP6 | 1051 | 1150 | 806 | 952 | 1 | 39 | 40 | 189 | 190 | 345 |
| Zm_AFP73 | 1052 | 1151 | 807 | 953 | 1 | 90 | 91 | 246 | 247 | 314 |
| Zm_AFP10 | 1053 | 1152 | 808 | 954 | 1 | 99 | 100 | 246 | 247 | 318 |

### Example 12. Transgenic Soybean Plants Expressing 1D Defensin have Enhanced Disease Resistance to Phakopsora Pachyrhizi

Transgenic soybean plants expressing 1D defensins listed in Table 17 were established and grown in soil in growth chambers and inoculated at the V2 stage with a pathogen suspension containing soybean rust pathogen, Phakopsora pachyrhizi. The plants were scored 12-14 days after inoculation for percent leaf infection. Disease reduction is reported as percent disease relative to the negative control (plants transformed with an empty vector) in Table 17. These results show that the transgenic soybean plants expressing 1D defensin, Cl.AFP44, ERAte_AFP29, or ERAte_AFP30 have enhanced resistance to Phakopsora pachyrhizi.

TABLE 17
Disease testing for trangenic soybean plants expressing 1D defensins

| | | | % disease | |
|---|---|---|---|---|
| | | | relative to | Statistically |
| | NUC SEQ ID NO/ | | negative | Significant |
| Tests | PRT SEQ ID NO | Locus ID | control | Activity? |
| 1 | 991/1090 | C1.AFP44 | 31 | yes |
| 2 | 991/1090 | C1.AFP44 | 38 | yes |
| 3 | 986/1085 | ERAte_AFP29 | 71 | yes |
| 4 | 987/1086 | ERAte_AFP30 | 65 | yes |

### Example 13. Transgenic Soybean Plants Expressing PINSY.AFP1 have Enhanced Disease Resistance to Phakopsora Pachyrhizi

Transgenic soybean plants expressing a codon optimized PINSY.AFP1 gene (SEQ ID NO: 1157) under the control of a synthetic promoter as set forth in SEQ ID NO: 1158 were generated. Field trials of transgenic plants expressing PINSY.AFP1 (polypeptide SEQ ID NO: 1107) were conducted to assess plant resistance to Phakopsora pachyrhizi utilizing multiple planting dates. The field trials were conducted using a randomized complete block design for each of two planting dates. Efficacy parameters were scored throughout the field trial season and at the end of the season yield was determined. Trait efficacy was measured by scoring disease prevalence 3 times at 14 day intervals once ASR was observed in the wildtype plots. Disease Ratings were taken as a 1-9 rating of overall plot infection (RST) and as a percent disease infection in the plots (DPPF). Efficacy data was utilized to calculate area under the disease progress curve (AUDPC) to determine overall disease resistance of each event. Data are presented in Table 18.

TABLE 18
Field Trial Efficacy Results as Rated disease infection (RST)
and Percent disease infection reduction (DPPF)

| | Area under disease | Area under disease | RST | DPPF |
|---|---|---|---|---|
| Event | progress curve RST | progress curve DPPF | LSD(.10) | LSD(.10) |
| Non-transgenic control | 31.9 | 319.4 | 7.4 total | 99.7 total |
| Transgenic Event 1 | 26.3 | 117.7** | 7.4 total | 99.7 total |
| Transgenic Event 2 | 21.0** | 95.4** | 7.4 total | 99.7 total |
| Transgenic Event 3 | 26.3 | 207.8* | 7.4 total | 99.7 total |
| Transgenic Event 4 | 22.8** | 163.6** | 7.4 total | 99.7 total |

*Significant reduction in disease at p ≤ 0.1
**Significant reduction in disease at p ≤ 0.05

The average rated disease for the control plants was 31.9. The average rated crop injury by disease for transgenic PINSy.AFLP1 soybean Event 1, Event 2, Event 3, and Event 4 was 26.3, 21.0, 26.3, and 22.8 respectively. The rated disease reduction least significant difference (LSD) at 0.10 was 7.4 for all events tested. The average percent disease for the non-transgenic control utilizing Area Under the Disease Progress Curve (AUDPC) 319.4. The average AUDPC for percent crop injury by disease for transgenic PINSy.AFP1 soybean Event 1, Event 2, Event 3, and Event 4 was 117.7, 95.4, 207.8 and 163.6 respectively. The AUDPC percent disease reduction least significant difference (LSD) at 0.10 was 99.7 for all transgenic events tested. These results indicate that all transgenic soybean events expressing PINSY.AFP1 have enhanced disease resistance to Phakopsora pachyrhizi.

### Example 14. Transgenic Corn Plants Expressing 1D Defensins have Enhanced Disease Resistance

Transgenic corn plants expressing 1D defensin were generated and grown in the greenhouse. At the VT growth stage, two nodes were infected by wounding the stalk and injecting a suspension of Colletotrichum graminicola inoculum into the wound. Corn plants were evaluated ˜2-3 weeks after inoculation. Stalks were harvested, leaves were removed and the stalks were split longitudinally. Disease severity was reported as the percent necrosis of the cut surface and is compared to the percent necrosis of non-transformed plants as shown in Table 19. Transgenic corn plants expressing defensin AMBtr_AFP7, Cl_AFP22, Cl_AFP44, ERAte_AFP95, Ph_AFP2, or PINsy_AFP1 as shown in Table 19 have enhanced disease resistance to Colletotrichum graminicola. Transgenic corn plants expressing defensin Ph_AFP2 were further tested for resistance to other pathogens and were shown to have enhanced broad-spectrum disease resistance to S. maydis F. verticiliodes, F. graminearum.

TABLE 19
Transgenic corn plants expressing 1D defensins have enhanced disease resistance

| | | | Number of events with significant |
|---|---|---|---|
| | | | disease reduction/number of |
| Defensin | NUC SEQ ID NO | PRT SEQ ID NO | events tested (at p-value < 0.2) |
| AMBtr_AFP7 | 961 | 1060 | 5 of 7 |
| Cl_AFP22 | 990 | 1089 | 3 of 7 |
| Cl_AFP44 | 996 | 1095 | 3 of 20 |
| ERAte_AFP95 | 1004 | 1103 | 6 of 9 |
| Ph_AFP2 | 1010 | 1109 | 9 of 17 |
| PINsy_AFP1 | 1108 | 1107 | 4 of 9 |

As various modifications could be made in the constructions and methods herein described and illustrated without departing from the scope of the invention, it is intended that all matter contained in the foregoing description or shown in the accompanying drawings shall be interpreted as illustrative rather than limiting. The breadth and scope of the invention should not be limited by any of the above-described exemplary embodiments, but should be defined only in accordance with the following claims appended hereto and their equivalents. All patent and non-patent documents cited in this specification are incorporated herein by reference in their entireties.

What is claimed is:

1. A recombinant DNA construct comprising a nucleic acid sequence encoding a multi-domain defensin polypeptide comprising a first defensin region connected to a second defensin region by a linker region, the first defensin region and the second defensin region each comprising a gamma-thionin domain, wherein the nucleic acid sequence encoding the multi-domain defensin polypeptide is operably linked to a promoter functional in a plant cell.

2. The recombinant DNA construct of claim 1, wherein the first defensin region is heterologous with respect to the second defensin region or the linker region.

3. The recombinant DNA construct of claim 1, wherein the first defensin region is identical to the second defensin region.

4. The recombinant DNA construct of claim 1, wherein the first defensin region is different from the second defensin region.

5. The recombinant DNA construct of claim 1, wherein the first defensin region or the second defensin region comprises a polypeptide having at least 80% identity to an amino acid sequence selected from the group consisting of SEQ ID NOs: 559-662.

6. The recombinant DNA construct of claim 5, wherein the first defensin region or the second defensin region comprises a polypeptide having an amino acid sequence selected from the group consisting of SEQ ID NOs: 559-662.

7. The recombinant DNA construct of claim 1, wherein the linker region comprises a polypeptide having at least 80% identity to an amino acid sequence selected from the group consisting of SEQ ID NOs: 153-202.

8. The recombinant DNA construct of claim 7, wherein the linker region comprises a polypeptide having an amino acid sequence selected from the group consisting of: SEQ ID NOs: 153-202.

9. The recombinant DNA construct of claim 1, wherein the multi-domain defensin polypeptide comprises an amino acid sequence having at least 80% identity to an amino acid sequence selected from the group consisting of SEQ ID NOs: 52-102, 329-454 and 1156.

10. The recombinant DNA construct of claim 9, wherein the multi-domain defensin polypeptide comprises an amino acid sequence selected from the group consisting of SEQ ID NOs: 52-102, 329-454 and 1156.

11. The recombinant DNA construct of claim 1, wherein the first defensin region or the second defensin region comprises an amino acid sequence having at least 80% identity to a sequence selected from the group consisting of SEQ ID NOs: 1054-1152 and 1154.

12. The recombinant DNA construct of claim 11, wherein the first defensin region or the second defensin region comprises a sequence selected from the group consisting of SEQ ID NOs: 1054-1152 and 1154.

13. The recombinant DNA construct of claim 1, wherein the multi-domain defensin polypeptide encoded by nucleic acid sequence further comprises an N-terminal transit signal sequence having at least 80% identity to an amino acid sequence selected form the group consisting of SEQ ID NOs: 809-954.

14. A plant, seed, plant tissue, plant part, or cell comprising the recombinant DNA construct of claim 1.

15. The plant, seed, plant tissue, plant part, or cell of claim 14, comprising the multi-domain defensin polypeptide encoded by the recombinant DNA construct of claim 1.

16. The plant, seed, plant tissue, plant part, or cell of claim 15, wherein the plant, seed, plant tissue, plant part, or cell has tolerance or activity against at least one plant fungal pathogen selected from the group consisting of Fusarium, Collectotrichum, Stenocarpella, and Phakopsora.

17. The plant, seed, plant tissue, plant part, or cell of claim 16, wherein the plant, seed, plant tissue, plant part, or cell has tolerance or activity against at least one fungal species selected from the group consisting of Fusarium graminearum, Fusarium verticilloides, Collectotrichum graminicola, Stenocarpella maydis, and Phakopsora pachyrhizi.

18. A microorganism comprising the recombinant DNA construct of claim 1.

19. A DNA molecule or vector comprising the recombinant DNA construct of claim 1.

20. The DNA molecule or vector of claim 19, wherein the nucleic acid sequence comprises a polynucleotide sequence having at least 70% identity to a nucleotide sequence selected from the group consisting of SEQ ID NOs: 1-51, 203-328 and 1155.

21. The DNA molecule or vector of claim 19, wherein the nucleic acid sequence comprises a polynucleotide having a sequence selected from the group consisting of SEQ ID NOs: 1-51, 203-328 and 1155.

22. A method for conferring fungal pathogen tolerance or resistance to a plant, seed, cell, or plant part comprising expressing in said plant, seed, cell, or plant part the multi-domain defensin polypeptide encoded by the recombinant DNA construct of claim 1.

23. A method for producing a transgenic plant with tolerance to a fungal pathogen comprising transforming a plant cell or tissue with the recombinant DNA molecule or vector of claim 18, and regenerating a transgenic plant.

24. A recombinant DNA construct comprising a nucleic acid sequence encoding a defensin polypeptide having at least 80% identity to an amino acid sequence selected from the group consisting of SEQ ID NOs: 1054-1152, wherein the nucleic acid sequence encoding the defensin polypeptide is operably linked to a promoter functional in a plant cell.

25. The recombinant DNA construct of claim 24, wherein the defensin polypeptide has an amino acid sequence selected from the group consisting of SEQ ID NOs: 1054-1152.

26. The recombinant DNA construct of claim 24, wherein the promoter comprises a nucleotide sequence as set for in SEQ ID NO: 1158.

27. A plant, seed, plant tissue, plant part, or cell comprising the recombinant DNA construct of claim 24.

28. The plant, seed, plant tissue, plant part, or cell of claim 27, wherein the plant, seed, plant tissue, plant part, or cell has tolerance or activity against at least one plant fungal pathogen selected from the group consisting of Fusarium, Collectotrichum, Stenocarpella, and Phakopsora.

29. The plant, seed, plant tissue, plant part, or cell of claim 28, wherein the plant, seed, plant tissue, plant part, or cell has tolerance or activity against at least one fungal species selected from the group consisting of Fusarium graminearum, Fusarium verticilloides, Collectotrichum graminicola, Stenocarpella maydis, and Phakopsora pachyrhizi.

30. A method for producing a transgenic plant with tolerance to a fungal pathogen comprising transforming a plant cell or tissue with the recombinant DNA molecule or vector of claim 24, and regenerating a transgenic plant.
